(19) United States
(12) Patent Application Publication
(10) Pub. No.: US 2015/0044672 A1
(43) Pub. Date: Feb. 12, 2015

(54) STREPTAVIDIN COMPLEXES AND USES THEREOF

(71) Applicants: Milan Stojanovic, Fort Lee, NJ (US); Steven Taylor, Jersey City, NJ (US)

(72) Inventors: Milan Stojanovic, Fort Lee, NJ (US); Steven Taylor, Jersey City, NJ (US)

(73) Assignee: The Trustees of Columbia University in the City of New York, New York, NY (US)

(21) Appl. No.: 14/343,741

(22) PCT Filed: Sep. 7, 2012
(86) PCT No.: PCT/US12/54250

§ 371 Date: Oct. 30, 2014

Related U.S. Application Data
(60) Provisional application No. 61/532,978, filed on Sep. 9, 2011.

(51) Int. Cl. C12Q 1/68

(52) U.S. Cl. CPC C12Q 1/6876
USPC 435/6.11

(57) ABSTRACT

Provided are compositions including a streptavidin composition in which a plurality of biotin binding sites are blocked by tethered biotins. Also provided are methods of using such compositions, including cell imaging, nucleic acid analysis or detection, or biotinylation quantification.

### CROSS-REFERENCE TO RELATED APPLICATIONS

The present application is a Continuation in Part of International Application No. PCT/US12/54250, filed 7 Sep. 2012; which claims the benefit of U.S. Provisional Application Ser. No. 61/532,978 filed 9 Sep. 2011, each of which are incorporated herein by reference in its entirety.

### STATEMENT REGARDING FEDERALLY SPONSORED RESEARCH OR DEVELOPMENT

This invention was made with government support under grant number CCF-0829744 awarded by the National Science Foundation and grant number 5RC2CA147925 awarded by the National Institutes of Health. The government has certain rights in the invention.

### MATERIAL INCORPORATED-BY-REFERENCE

The Sequence Listing, which is a part of the present disclosure, includes a computer readable form comprising nucleotide and/or amino acid sequences of the present invention. The subject matter of the Sequence Listing is incorporated herein by reference in its entirety.

### FIELD OF THE INVENTION

The present disclosure generally relates to streptavidin complexes with oligodentate biotin displaying ligands.

### BACKGROUND OF THE INVENTION

Streptavidin is a 53 kDa protein homotetramer with four biotin binding pockets formed at each of the four monomer-monomer interfaces. One streptavidin can bind four molecules of biotin. The streptavidin-biotin interaction demonstrates extraordinary stability (e.g., with respect to heat, pH, denaturants, proteolysis) and selectivity. The streptavidin-biotin complex has a strong binding affinity (e.g., Kd˜10⁻¹³ to 10⁻¹⁴ M). The resulting streptavidin-biotin complex is effectively irreversible under physiological conditions. Numerous commercially available biotin- and streptavidin-conjugates. Applications include: biosensors, molecular beacons, clinical diagnostics, imaging, purification, and immunohistochemistry (IHC). Fusion proteins can be expressed with “biotin acceptor peptides” that can be biotinylated by biotin ligase.

The streptavidin-biotin complex is widely exploited in numerous assays in biotechnology, biomedical and analytical applications, and also has increasing uses in nanotechnology, due to its robustness, ease of use, and the foundation that many compositions can be functionalized with biotin or streptavidin or both. But non-monovalent streptavidin can induce membrane protein aggregation from wild-type streptavidin that can alter protein function. Protein aggregation and resulting functional alteration can cause significant problems when imaging proteins in live cells.

Genetically engineered monovalent streptavidin with a single biotin binding site has been reported in live cell imaging (see e.g., Howarth and Ting 2006 Nature Protocols 3(4), 267-273). Analyzing oligonucleotides, such as microRNAs, has been reported (see e.g., Qavi et al. 2010 Anal Bioanal Chem 398, 2535-2549). A tridentate biotin in which three biotins do not bind to the same streptavidin has been reported (see e.g., FIG. 15; Tei et al, Chem. Eur. J. 2010, 16, 8080-8087; Wilbur et al., Bioconjugate Chem. 1997, 8, 819-832).

Biotinylation of proteins (and other target structures, e.g., nanoparticles) is a widely used functionalization reaction, and is a cornerstone in a large number of bioassays, purification procedures, and imaging protocols. Whilst there are biotinylated-targets (e.g., biotinylated-proteins) commercially available, many end users want or need biotinylate a target of interest. But a biotinylation reaction can be difficult to quantify success or degree. It can be important to control the extent of biotinylation as over-biotinylation can result in inactivity of a target (e.g., a protein) of interest or destruction of function. Over-biotinylation can also lead to precipitation or loss of target (e.g., a protein). On the other hand, under-biotinylation can result in poor yields of biotinylated target (e.g., protein). Conventionally, the process of biotinylation can be one of trial and error, with each different target (e.g., protein) requiring a different set of conditions. Thus is provided herein an easy, quick, or accurate method to determine biotinylation results so as to optimize a process or to minimize costs.

Most all commercially available biotinylation reagents and kits use the HABA/avidin system to analyze the results of a biotinylation reaction. The disadvantages of the HABA/avidin system include: consumption of relatively large amounts of protein sample; requirement for a calibration curve; requirement for expensive equipment (e.g. UV-vis or fluorescence spectrometer); risk of under-representing the actual number of biotins covalently attached; restricted to potassium ion-free buffers; and does not work for all proteins, e.g. BSA. A commercially available alternative to the HABA/avidin system is a biotinylation reagent with an in-built signaling moiety, but this reagent is susceptibility to hydrolysis, which can result in the biotin moiety becoming detached from the protein.

### SUMMARY OF THE INVENTION

The present disclosure is based at least in part on the discovery that using a tridentate biotin ligand to block three of streptavidin's four biotin binding sites forms a highly stable one-to-one complex. Various embodiments provide a readily accessible monovalent streptavidin, assembled in a straigthforward procedure that can overcome a statistical distribution of products and substitutes protein expression with two off-the-shelf reagents (e.g., streptavidin and custom made oligonucleotide). Such reagent can be finely tuned with various functionalities, using a wide variety of custom analogs available for synthetic oligonucleotides. The unique properties of the complex and ease of synthesis opens wide opportunities for practical applications in, for example, imaging and biosensing.

One aspect provides a composition including a streptavidin molecule comprising four biotin binding sites; at least two biotin molecules; and at least one linker. In some embodiments, the linker connects the first biotin and the second biotin. In some embodiments, the first biotin is bound to a first biotin binding site of the streptavidin. In some embodiments, the second biotin is bound to the second biotin binding site of the streptavidin.

Some embodiments of the composition further include a third biotin and a second linker. In some embodiments, the second linker connects the third biotin to one or more of the first biotin, the second biotin, or the first linker, and the third biotin is bound to a third biotin binding site of the streptavidin.

In some embodiments, none of the linked biotins bind to another streptavidin.

In some embodiments, the linker includes a nucleic acid, an organic compound, or a combination thereof. In some embodiments, the linker comprises a DNA, an RNA, a locked nucleic acid (LNA), an inaccessible RNA, a peptide nucleic acid (PNA), or a combination thereof.

In some embodiments, the linker is (i) at least about 1.8 nm or (ii) at least about 6 nm in length. In some embodiments, the linker is at least about 1.8 nm, at least about 1.9 nm, at least about 2.0 nm, at least about 2.5 nm, at least about 2.6 nm, at least about 2.7 nm, at least about 2.8 nm, or at least about 2.9 nm in length. In some embodiments, the linker is at least about 6 nm, at least about 7 nm, at least about 8 nm, at least about 9 nm, at least about 10 nm, at least about 11 nm, at least about 12 nm, at least about 13 nm, at least about 14 nm, at least about 15 nm, at least about 16 nm, at least about 17 nm, at least about 18 nm, at least about 19 nm, or at least about 20 nm in length.

In some embodiments, the first linker is at least about 1.8 nm, at least about 1.9 nm, at least about 2.0 nm, at least about 2.5 nm, at least about 2.6 nm, at least about 2.7 nm, at least about 2.8 nm, or at least about 2.9 nm in length and (ii) the second linker is at least about 6 nm, at least about 7 nm, at least about 8 nm, at least about 9 nm, at least about 10 nm, at least about 11 nm, at least about 12 nm, at least about 13 nm, at least about 14 nm, at least about 15 nm, at least about 16 nm, at least about 17 nm, at least about 18 nm, at least about 19 nm, or at least about 20 nm in length.

In some embodiments, the streptavidin has an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, or at least about 95% identical thereto and retaining or substantially retaining a high affinity for biotin.

One aspect provides a method of detection that includes contacting a streptavidin/biotin composition as described above and a biotinylated target molecule under conditions where a biotin binding site of the composition can bind to the biotin of the biotinylated target molecule. In some embodiments, the method includes detecting the composition bound to the biotinylated target molecule. In some embodiments, the target molecule is attached to the surface of a cell. In some embodiments, the composition comprises a detectable tag. In some embodiments, the target molecule comprises an amino acid or a nucleic acid.

One aspect provides a method of detecting a nucleic acid in a sample that includes providing streptavidin/biotin composition described above, wherein the composition includes a streptavidin, at least three biotin molecules, a first linker, and a second linker comprising a nucleic acid complementary or substantially complementary to at least a portion of a target nucleic acid compound; combining the composition with a sample that may contain the target nucleic acid compound under conditions that, if the target nucleic acid compound is present, the target nucleic acid binds to the complementary nucleic acid linker resulting in at least one biotin being dislodged from the streptavidin thereby exposing a streptavidin-biotin binding site; contacting a labeled streptavidin with the composition; and detecting presence or absence of the label; wherein presence of the label indicates presence of the target nucleic acid compound in the sample.

In some embodiments, the target nucleic acid is a microRNA. In some embodiments, the microRNA is about 20 to about 25 nucleotides in length. In some embodiments, the microRNA is about 20, about 21, about 22, about 23, about 24, or about 25 nucleotides in length. In some embodiments, the nucleic acid of the second linker is the same or substantially the same length as the microRNA.

In some embodiments, if the target nucleic acid fully matches the nucleic acid of the second linker, the biotin binding site is exposed. In some embodiments, if one or more mismatches exist between the target nucleic acid and the nucleic acid of the second linker, the biotin binding site is not exposed. In some embodiments, if the target nucleic acid fully matches the nucleic acid of the second linker, the biotin binding site is exposed and if one or more mismatches exist between the target nucleic acid and the nucleic acid of the second linker, the biotin binding site is not exposed.

Another aspect provides a claim 17. A method of determining biotinylation of a target molecule. In some embodiments, the method includes providing a sample comprising a biotinylated target molecule, combining the sample and a composition according to claim 1 comprising monovalent streptavidin under conditions where the one available biotin binding site of the streptavidin in the composition can bind to a biotin of the biotinylated target molecule in the sample, separating biotinylated target molecules according to differing numbers of monovalent streptavidin bound thereto, and determining biotinylation level of the target molecule.

In some embodiments, the biotinylated target molecule and the monovalent streptavidin are combined in a ratio of at least about 1:1, about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, or about 1:20. In some embodiments, there are a plurality of samples comprising a biotinylated target molecule, at least a portion of which are combined with different amounts of monovalent streptavidin, and the biotinylated target molecules are separated according to differing numbers of monovalent streptavidin bound thereto. In some embodiments, the polyacrylamide gel electrophoresis (PAGE) is used to separate biotinylated target molecules according to differing numbers of monovalent streptavidin bound thereto.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1A illustrates assembly of a discrete tris-biotinylated oligonucleotide-streptavidin complex: streptavidin is represented by grey rectangle with a ‘bite’ out of each corner representing each biotin binding pocket; biotin as black spheres; a single stranded oligonucleotide and organic spacers link the three biotin moieties.

FIG. 1B is an image of a Native PAGE. Lane 1 is purified STV-(1); Lanes 2-4 are a mixture of STV-(1) with 0.1, 0.5 and 2 equivalents of a monobiotinylated oligonucleotide resulting in a single major product—thus supporting that STV-(1) is monovalent; Lane 5 is the monobiotinylated oligonulceotide; Lane 6 is the fluorescent dye labelled oligonucleotide Cy3-A25-Cy3; Lane 7 is a mixture of STV-(1) and Cy3-A25-Cy3 resulting in a product ‘ladder’ due to oligomer of varying lengths.

FIG. 2 shows HPLC traces for yield optimization with desthiobiotin, and anion exchange HPLC purification (TSK column) of STV-(2) (where (2) is 5′-dualbiotin-24 mer-monobiotin-3′).

FIG. 3 shows anion exchange HPLC results for the assembly and characterization of STV-(2): (i) baseline. (ii) STV-(GAC TAT CGC CTT CAT ACT ACC TCC-monobiotin-3′)ₙ “ladder” where n=1, 2, 3, and 4. (iii-v) Assembly and HPLC purification of STV-(2): (iii) (2); (iv) 1×STV+1×(2) (by-products are circled by broken line}. (v) Purified STV-(2). (vi-viii) Titration of purified STV-(2) with GAC TAT CGC CTT CAT ACT ACC TCC-monobiotin-3′ (i.e. 1^{†}) demonstrating the “monovalent” nature of STV-(2): (vi) 1×STV-(1)+0.5(1^{†}); (vii) 1×STV-B₃+2(1^{†}); (viii) (1^{†}).

FIG. 4A shows HPLC results from assembly and purification of STV-B₃ (top trace=1×STV+1×B₃ at 25° C. followed by heating at 70° C. for 15 mins; Second to top trace=1×STV+1×B₃ at 25° C.; middle trace=1×STV+2×B₃ at 25° C.; second to lowest trace=baseline; lowest trace=STV-(5′-biotin-T25)ₙ standard markers, where n is 1, 2, 3 or 4.

FIG. 4B shows HPLC results for titration of purified STV-B3 with a 5′-monobiotin-50 mer oligonucleotide: from lowest trace-to-highest trace: STV-(5′-biotin-T25)n standard markers; baseline; STV-B3; STV-B3+0.5×5′-monobiotin-50 mer; STV-B3+1×5′-monobiotin-50 mer; STV-B3+2×5′-monobiotin-50 mer.

FIG. 4C shows HPLC results for stability of STV-B3 at 70° C. in the presence of excess biotin in comparison with STV-(5′-biotin-T25)n.

FIG. 4D shows STV-B3 and B1-STV-B3 (ring closed products).

FIG. 5 shows non-denaturing PAGE results. Addition of sub-(1% and 10%) and excess equivalents (1000%) of oligomerization triggering oligonucleotide Cy3A25Cy3 to STV(1).

FIG. 6 shows a non-denaturing PAGE demonstrating biotin-streptavidin dissociation (compare lanes 3 and 4 with 2) and prevention of biotin-streptavidin dissociation by incorporating a fourth biotin on the biotinylated oligonucleotide (compare lane 6 with lane 2): Lane 1 is pure STV-(2); Lane 2 is STV-(2) with two equivalents 24-mer full complement; Lanes 3 and 4 are analogous to Lane 2 except that excess biotin or streptavidin repectively are present. Lanes 5-8 are analogous to lanes 1-4 except that STV-(4) is used in place of STV-(2).

FIG. 7 shows native PAGE results for the effect of complimentary strand length (N) on the extent of oligomerization after 1 hour incubation at room temperature. Lane “0” is no complementary strand added; Lane “S” is 24 mer complimentary strand added in the presence of 10 equivalents of streptavidin; Lane “B” is 24 mer complimentary strand added in the presence of 1000 equivalents of biotin.

FIG. 8 shows non-denaturing PAGE results of addition of perfectly matched or single mismatch oligonucleotide to STV-(3) compared with F-STV-(3) (where the lone vacant biotin-binding site is blocked with fluorescein-biotin (•-F)) after 15 mins incubation at 37° C.

FIG. 9 shows non-denaturing PAGE results of single mismatch sensitivity of (a) STV-(2), and (b) STV-(3) for all possible NN base-base mismatch combinations of N=dG, dC, dA, or dT at selected positions of a 17 mer oligonucleotide complement, after 15 minutes incubation at ambient temeperature (Non-denaturing PAGE: 4% stacking layer run at 100V, 10% seperation layer run at 200V). The lower half of each gel which showed single bands of non-duplexed complementary strand has been cut off for the purpose of layout. Lanes labeled 1 are STV-(2) or STV-(3) and lanes labeled 2 are STV-(2) or STV-(3) plus addition of two equivalents of perfectly matched 17 mer complementary oligonucleotide. All other lanes for a and b contain a compliment 17 mer oligonucleotide with a single mismatch. (c) Key to mismatch positions. (d) Using STV-(2) “b” refers to the presence of excess biotin, and “s” refers to the presence of excess streptavidin; Lanes 12 and 13 are STV-(2)+0.01 equivalents and +2 eqivalents of 24 mer complement oligonucleotide respectively. (e). *Analogous to a except samples were incubated for a further 72 hours.

FIG. 10 shows PAGE results for the effect of a single mismatch on the biotin dissociation process at room temperature. Lanes marked with a “k” contains the single mismatch oligonucleotide. “-ve” refers to STV-(2) control, i.e. no complement strand added. Mixture was incubated at room temperature for 15 mins in 20 mM TRIS pH 7.2, 150 mM NaCl. The initial mismatches were selected to occur near as possible at a position in the middle of the target oligonucleotide. For (M)Nmer (where M indicates the mono-biotinylated end of the oligonucleotide) complement strands, an AA mismatch at position 11 was used for N=20; TT at position 10 for N=19, and AA at position 9 for N=16, 17, and 18. For (D)Nmer (where D indicates the dual-biotinylated end of the oligonucleotide) complement strands, a CC mismatch at position 10 for N=19 and 20, and a CC mismatch at position 9 N=16, 17 and 18, was incorporated. Results for (M)Nmers showed significant sensitivity, i.e. a higher biotin dissociation rate, only for the (M)16 mer*. For >M16 mers, sensitivity quickly diminished. In contrast, results for (D)Nmers showed great sensitivity for all lengths (D)16 mer-to-(D)20 mer. One explanation for this difference between M and D could be the slightly higher percentage of GC base-pairing that occurs in M over D i.e. a one additional GC pair difference.

FIG. 11 shows native PAGE results for single mismatch sensitivity of F-STV-(3) for all possible NN base-base mismatch combinations of N=dG, dC, dA, or dT at selected positions of a 17 mer oligonucleotide complement, after 15 minutes incubation at 37° C. (Native PAGE). Bands due to the 2-fold excess of compliment added are obscured in the darken area due to camera exposure settings chosen to maximize the intensity of all minor bands.

FIG. 12 shows ELISA results for: a—QuantaRed HRP substrate only; b—positive control, i.e. biotin present; c—perfect match; d—single base mismatch; e—negative control i.e. no compliment added.

FIG. 13 shows an ELISA 96-well plate. Wells A1-A5 are described in Example 1 and FIG. 12. Wells B1-B5 have 10-fold less (5), and 10-fold less (6) or STV-(3) added relative to row A wells. Wells C1-C5 have 100-fold less (5), and 100-fold less (6) or STV-(3) added relative to row A wells.

FIG. 14 shows a cartoon whereby perfectly matched oligonucleotides trigger dissociation of the biotin-streptavidin interaction at higher rates relative to SNPs.

FIG. 15 is a scheme that illustrates the use of a biotin linker to block three of four biotin binding sites on streptavidin by mixing equimolar amounts of linker to streptavidin. FIG. 15 also illustrates that by providing a complementary oligonucleotide to a single strand of the oligonucleotide linker can pull off one biotin, freeing a binding site on the streptavidin, where the free biotin end can bind with a streptavidin with a label. FIG. 15 also depicts that the longest arm of the tridentate biotin can be an organic linker.

FIG. 16 shows one embodiment of streptavidin-B3 microRNA detection.

FIG. 17 shows dimensions of streptavidin and the tris-biotinylated oligonucleotide. (a) The four bound biotin molecule's carboxyl group oxygen atoms (the point of attachment for all biotinylated molecules) form a distorted rectangular plane with approximate dimensions of (3.1×1.9)nm with a dihedral angle of 25°. The two short-side biotins can be tethered together by a linker measuring at least 1.9 nm. The two longer side biotins are 3.1 nm apart directly through the protein, but linkers need to be greater than 6.0 nm to reach around the protein. (b) A single stranded 25 nucleotide oligonucleotide. (c) A double stranded 25 nucleotide helix. All of a, b, and c are to scale.

FIG. 18. shows assembly of a “monovalent streptavidin” species (highlighted by circle). Grey is streptavidin, black circles are biotin, two biotins are connected by an organic spacer, which are connected to the third biotin via an oligonucleotide. Far Right: A fluorescently labeled “monovalent streptavidin” species attached to a cell surface protein by a SINGLE biotin. The tetravalent streptavidin shown is cross-linking two cell surface proteins which can cause disruption of normal cellular processes.

FIG. 19 shows an illustration of STV-B3* (ring-closed)-T25 sequence.

FIG. 20A shows PAGE results for the effect of target strand length on the biotin dissociation process at room temperature. Target and probe were mixed and incubated at room temperature for at least 15 mins in 20 mM TRIS, 150 mM NaCl, pH 7.2. FIG. 20B shows PAGE results for the effect of target strand length on the biotin dissociation process at room temperature. Target and probe were mixed and incubated at room temperature for at least 15 mins in 20 mM TRIS, 150 mM NaCl, pH 7.2. FIG. 20C shows PAGE results for the effect of target strand length on the biotin dissociation process at room temperature. Target and probe were mixed and incubated at room temperature for at least 15 mins in 20 mM TRIS, 150 mM NaCl, pH 7.2. FIG. 20D shows PAGE results for the effect of target strand length on the biotin dissociation process at room temperature. Target and probe were mixed and incubated at room temperature for at least 15 mins in 20 mM TRIS, 150 mM NaCl, pH 7.2.

FIG. 21A shows PAGE results for the effect of a single mismatch on the biotin dissociation process at room temperature. Lane marked with a “*” contains single mismatch oligonucleotides. “-ve” refers to STV-B3 control, i.e. no target added. Target and probe were mixed and incubated at room temperature for 15 mins in 20 mM TRIS, 150 mM NaCl, pH 7.2. FIG. 21B shows PAGE results for the effect of a single mismatch on the biotin dissociation process at room temperature. Lane marked with a “*” contains single mismatch oligonucleotides. “-ve” refers to STV-B3 control, i.e. no target added. Target and probe were mixed and incubated at room temperature for 15 mins in 20 mM TRIS, 150 mM NaCl, pH 7.2. FIG. 21C shows PAGE results for the effect of a single mismatch on the biotin dissociation process at room temperature. Lane marked with a “k” contains single mismatch oligonucleotides. “-ve” refers to STV-B3 control, i.e. no target added. Target and probe were mixed and incubated at room temperature for 15 mins in 20 mM TRIS, 150 mM NaCl, pH 7.2. FIG. 21D shows PAGE results for the effect of a single mismatch on the biotin dissociation process at room temperature. Lane marked with a “k” contains single mismatch oligonucleotides. “-ve” refers to STV-B3 control, i.e. no target added. Target and probe were mixed and incubated at room temperature for 15 mins in 20 mM TRIS, 150 mM NaCl, pH 7.2.

FIG. 22 shows PAGE results for NN mismatch combinations (where N is G, C, A, or T). The mismatched base of the target strand is shown in red. The position that the mismatch occurs is given as a number in each lane and also indicated by a red arrow. Numbering starts at the 5′ position of the probe strand i.e. “B3*”. Target and probe were mixed and incubated at 37° C. for 15 mins in 20 mM TRIS, 150 mM NaCl, pH 7.2.

FIG. 23 shows oligonucleotide detection by ELISA using a Horseradish peroxidase tag (HP).

FIG. 24 illustrates reaction schemes with products.

FIG. 25 shows tagging biotin moieties with monovalent streptavidin and quantification thereof. FIG. 25A is a cartoon showing tagging of biotin moieties with monovalent streptavidin. FIG. 25B is an image of a Native PAGE (8%) gel with SYBR Gold staining, where the lanes include biotinylated bovine serum albumin tagged with monovalent streptavidin. Further information regarding methodology is available in Example 13.

FIG. 26 shows biotinylation of the therapeutic antibody Rituxan, tagging with monovalent streptavidin, and quantification thereof. FIG. 26A is a cartoon showing biotinylation of the therapeutic antibody Rituxan and tagging with monovalent streptavidin. FIG. 25B is an image of a Native PAGE (8%) gel with with SYPRO® Ruby protein gel stain, where each lane contains 10 pM of biotinylated antibody and 1 to 10 equivalents of monovalent streptavidin reagent. FIG. 26C is densitometry analysis showing distribution of biotinylated products from lanes of FIG. 1B.

### DETAILED DESCRIPTION OF THE INVENTION

The present disclosure is based at least in part on the discovery that using a tridentate biotin ligand to block three of streptavidin's four biotin binding sites forms a highly stable one-to-one complex. Furthermore, the present disclosure is based at least in part on the discovery that a monovalent streptavidin-oligonucleotide conjugate can be a sensitive sensor of single-point mutations—hybridization of a cyclized oligonucleotide with a perfectly matched complementary strand can trigger dissociation of the biotin-streptavidin interaction with concomitant oligomerization, and with more efficiency than a strand containing a single nucleotide polymorphism.

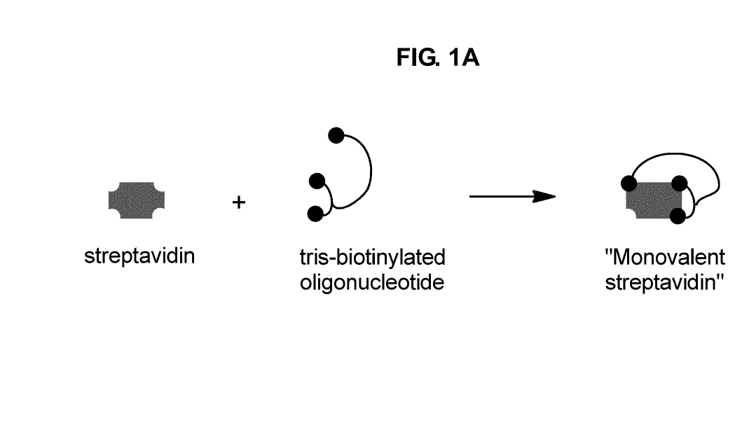

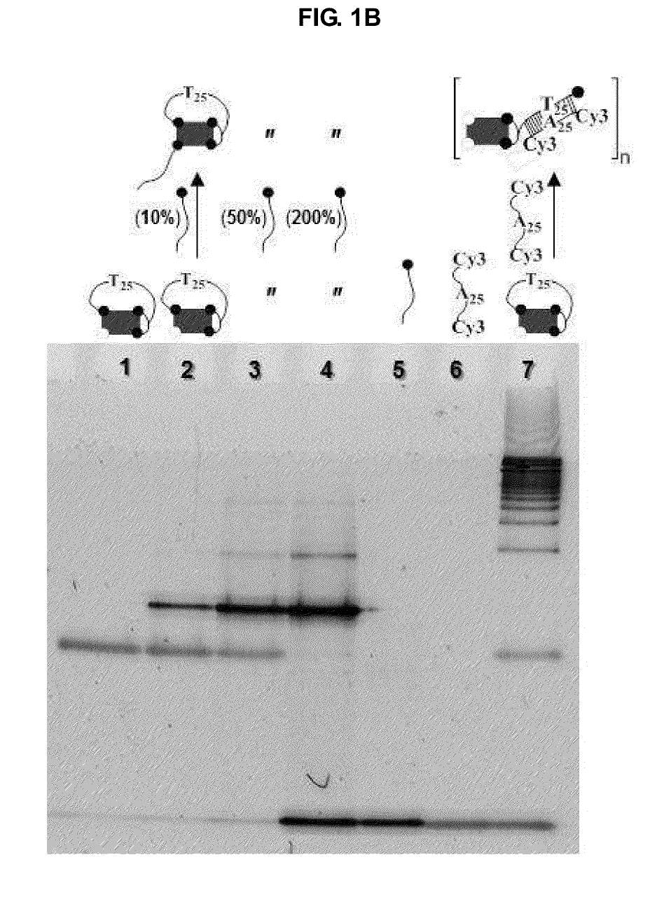

Provided herein are various embodiments of a straightforward single-step route to a “monovalent streptavidin” (streptavidin with only one biotin binding site), making use of the chelate effect to increase stability and yield using a tris-biotinylated-oligonucleotide to block three of streptavidin's four biotin-binding sites (see e.g., FIG. 1). The ligand can allow the straightforward generation of monovalent streptavidin which can be desirable in cell labeling applications where, for example, cross-linking is detrimental. For example, an oligonucleotide with three appended biotin moieties can bind preferentially in tridentate fashion to a single streptavidin at a ratio of one-to-one. Such a monovalent biotin can be used in applications such as imaging (e.g., fluorescence and radio) and can avoid crosslinking receptors on cell surfaces.

One solution to the problem of crosslinking can be to eliminate streptavidin binding sites. A streptavidin monomer binds biotin relatively weakly due to the binding pocket in tetrameric streptavidin occurring between monomers; hence monomeric streptavidin does not provide an effective way around unwanted cross-linking. A mutant streptavidin tetramer has been reported, where the streptavidin is engineered with only one functioning biotin binding pocket by producing mutations in the other three. Yet, this approach requires extensive effort to engineer and express the mutant streptavidin. In contrast, a “monovalent streptavidin” as described herein provides an alternative (and simpler) solution by preventing binding of biotin in three of streptavidin's four binding sites by blocking three sites with a tightly binding tridentate-biotin ligand (e.g., B3 ligand). For example, a monovalent streptavidin can be attached to a cell protein using the only open site to avoid bridging problems that can occur in native streptavidin. With B3 binding in multidentate fashion, the binding affinity of the ligand can be several orders of magnitude larger than an already tightly binding monodentate biotin ligand. This can make the complex highly resistant to biotin substitution.

Furthermore, applications such as biomolecule labeling, purification, immobilization, and patterning can be complicated by multimerization of wild-type streptavidin. A monovalent streptavidin can reduce or eliminate such complications.

An additional feature of a tridentate-biotin ligand (e.g., B3) is an increase to detection limit. A monovalent streptavidin-B3 conjugate can allow the triggering of oligomerization, which has the potential to lead to signal amplification in such applications as MRI or other techniques which inherently suffer from low detection limits.

One aspect provided herein is a monovalent streptavidin. A streptavidin can be made monovalent by the addition of a multi-dentate biotin (e.g., biotin 2-, 3-, and 4-mers). The biotin n-mers can bind to streptavidin to form STV-B complexes of varying degrees of valencies to number linking streptavidins. The STV-B complexes can also be “switched” from, for example, mono-valency to divalency.

Furthermore, the present disclosure is based at least in part on the discovery that one of three bound biotins can be facilely dissociated from its streptavidin complex by an addition of compound (e.g., an oligonucleotide) complementary to a linker linking the biotins into a single moiety. A tridentate-biotin ligand comprising an oligonucleotide can be used to block three of streptavidin's four biotin binding sites. The ligand can include three appended biotin moieties. One of the three bound biotins can be facilely dissociated from its streptavidin complex by inputting a complement oligonucleotide. The dissociation results in a change of ligand binding mode from tridentate chelation (to a single streptavidin) to bridging (between streptavidin molecules), leading to formation of streptavidin dimers and higher oligomers.

Such an inbuilt switch for biotin dissociation can provide a simple mechanism for oligonucleotide detection. Such dissociation can be used as a switch, where the presence of the complementary compound can be translated into the presence of a new biotin molecule. The biotin dissociation can be sensitive to single point polymorphisms (SNPs). Additionally, the ligand can allow the straightforward generation of a monovalent streptavidin, which is useful in cell labeling applications where cross-linking interferes with cell function. Uses of the switch include, but are not limited to, oligomerization of streptavidin or further cell labeling. Such a switch mechanism to reveal a biotin moiety can likewise be used in other streptavidin complexes, including but not limited to a bis-biotin/streptavidin complex.

Exposing a biotin or biotin binding site by a molecular triggering event can add a new dimension to the design of biotin/streptavidin based diagnostics and therapeutics. For example, given the large variety of methods currently available to convert a biotin “flag” into a detectable signal, a quick and sensitive one-step procedure, as described herein, which sends up a biotin flag in direct response to a specific oligonucleotide sequence (e.g., an miRNA) can offer several advantages over current detection methods which are often complex and time consuming. As an example, the Northern blot, a popular method for miRNA analysis, can take several days for complete analysis. The conventional RAKE assay for incorporating a biotin flag into detection of a target miRNA involves four steps between capture of the target and generation of a fluorescent signal—1) hybridization, 2) exonuclease treatment, 3) biotinylation, and 4) fluorophore conjugation. In contrast, using compositions and methods described herein, detection of a specific oligonucleotide sequence can be made in one simple 15 minute hybridization step (with, for example, single mismatch discrimination) followed by PAGE analysis.

Also provided is a process for forming a streptavidin having one, two, or three biotin binding sites blocked. In one embodiment, a simple one-step process is used to form a highly stable monovalent streptavidin species includes using a tridentate-biotin ligand to block three of streptavidin's four biotin binding sites. The ligand can be an oligonucleotide with three appended biotin moieties. One of the three bound biotins can be facilely dissociated from its streptavidin complex by inputting a complement oligonucleotide. Such an inbuilt switch for biotin dissociation can provide a simple mechanism for oligonucleotide detection. The biotin dissociation can be sensitive to single point polymorphisms (SNPs). Additionally, the ligand can allow the straightforward generation of a monovalent streptavidin, which is useful in cell labeling applications where cross-linking interferes with cell function. It has been demonstrated that monovalent streptavidin does not cause labeling-dependent aggregation as can occur with wild-type streptavidin. Analogous results were obtained for a bidentate version of the ligand.

Streptavidin

Various compositions and methods described herein can employ a streptavidin having one or more biotin sites blocked (e.g., three of four biotin binding sites blocked).

A streptavidin can be any protein having a high affinity for biotin (e.g., Kd of about 10⁻¹⁴ mol/L). A streptavidin or a nucleotide encoding such, can be isolated from the bacterium Streptomyces (e.g., Streptomyces avidinii). A streptavidin can be any commercially available streptavidin (e.g., Invitrogen; Qiagen; Thwermo Scientific; Jackson ImmunoResearch; Sigma Aldrich; Cell Signalling Technology). A streptavidin can be of an amino acid sequence according to Accession No. AAM49066.1; Accession No. YP_{—}001064618.1; Accession No. YP_{—}001081770.1; Accession No. YP_{—}001057375.1; Accession No. YP_{—}001028007.1; Accession No. YP_{—}438916.1; Accession No. YP_{—}440845.1; Accession No. YP_{—}104836.1; Accession No. ZP_{—}09081347.1; Accession No. EHI14260.1; Accession No. ACL82594.1; or Accession No. CAA00084.1.

A streptavidin can have an amino acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, or at least about 80% identical thereto and retaining or substantially retaining high affinity for biotin. For example, a streptavidin can have an amino acid sequence at least about 85%, at least about 90%, at least about 95%, at least about 96%, at least about 97%, at least about 98%, or at least about 99% identical to any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, and retaining or substantially retaining high affinity for biotin.

A streptavidin can be a naturally occurring streptavidin. For example, a streptavidin can be a naturally occurring streptavidin from Burkholderia spp. (e.g., B. pseudomallei, B. mallei, B. thailandensis), Mycobacterium spp. (e.g., M. thermoresistibile), or Streptomyces spp. (e.g., S. lavendulae, S. avidinii). A streptavidin can be a variant of a naturally occurring streptavidin having at least about 80%, 85%, 90%, 95%, or 99% sequence identity thereto and retaining or substantially retaining high affinity for biotin.

Biotin

Various compositions and methods described herein can employ multiple biotin molecules linked together and capable of blocking one or more biotin binding sites of a streptavidin.

A biotin can be a water soluble B-complex vitamin (e.g., vitamin B₇, coenzyme R, vitamin H). A biotin can be a heterocyclic sulfur-containing (mono-)carboxylic acid. A biotin can comprise an imidazole ring and thiophene ring fused. A biotin can comprise a ureido (tetrahydroimidizalone) ring fused with a tetrahydrothiophene ring, optionally with a veleric acid substituent on a carbon of the tetrahydrothiophene ring. A biotin can be any commercially available biotin (e.g., Invitrogen; Qiagen; Thwermo Scientific; Jackson ImmunoResearch; Sigma Aldrich; Cell Signalling Technology). A biotin can be a variant compound of a naturally occurring biotin that retains or substantially retaining high affinity for streptavidin.

A streptavidin can bind biotin with high affinity (e.g., Kd of 10⁻¹⁴ mol/l to 10⁻¹⁵ mol/l) and specificity.

A biotin can be any commercially available biotin (e.g., Invitrogen; Qiagen; Thwermo Scientific; Jackson ImmunoResearch; Sigma Aldrich; Cell Signalling Technology). A biotin can be a variant compound of a naturally occurring biotin that retains or substantially retaining high affinity for streptavidin.

A biotin can have a structural formula according to C₁₀H₁₆O₃N₂S. A biotin can have a structure as follows:

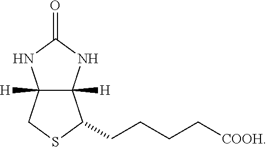 [SMILES: O=C(O)CCCC[C@@H]1SC[C@@H]2NC(=O)N[C@@H]21]

Linker

Various compositions and methods described herein can employ one or more ligands or linkers linking multiple biotin molecules together such that the resulting linked biotin composition can block one or more biotin binding sites of a streptavidin.

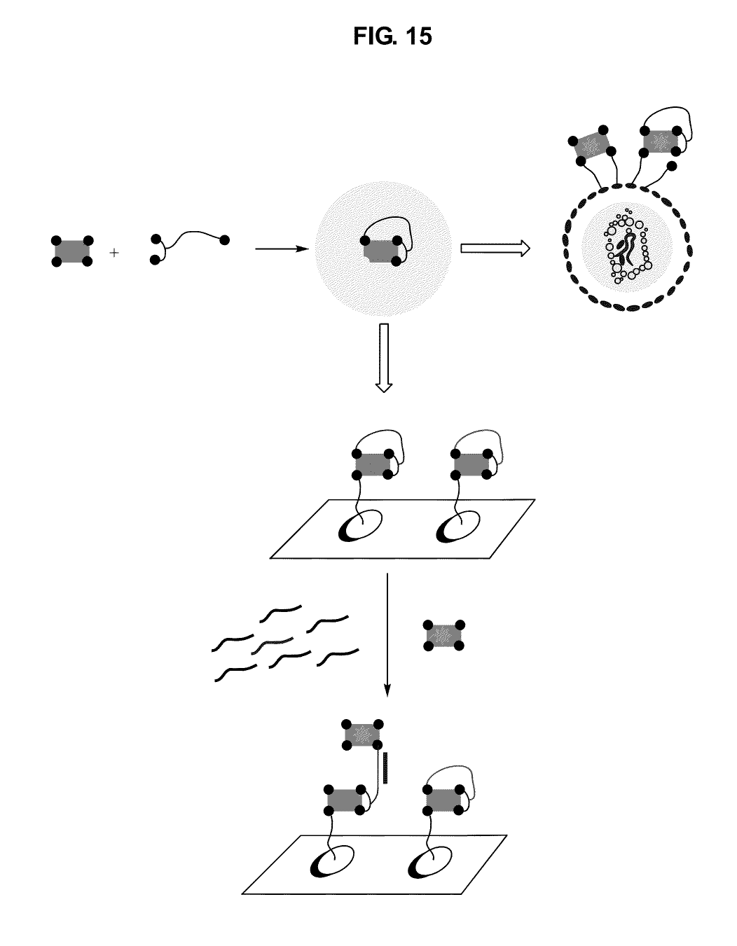

A tridentate biotin can be synthesized using a linker. A linker can include, for example, DNA, LNA, PNA, or organic linkers, or any combination thereof. As shown herein, a tridentate biotin linker blocked three quarters of streptavidin sites by mixing equimolar amounts of linker and streptavidin. Moreover, providing a complementary oligonucleotide to a single strand of the oligonucleotide linker can pull off one biotin, freeing a binding site on the streptavidin, where the free biotin end can bind with a streptavidin with a label. Also, the DNA for the longest arm can be an organic linker. Additionally, the monovalent streptavidin can be used to detect short oligonucleotides by choosing an appropriate linker (see FIG. 15).

A linker can include a material such as a nucleic acid (e.g., DNA or RNA), organic compounds, or a combination thereof. One composition can include a plurality of linkers, each of which can be independently selected. One linker can contain a plurality of materials.

A linker can comprise a locked nucleic acid (LNA) also known as locked sugars; an inaccessible RNA, which is a modified RNA nucleotide; or a peptide nucleic acid (PNA), or a combination thereof. PNA is similar to DNA or RNA, but is not known to occur naturally, and unlike DNA or RNA, has an uncharged backbone. A linker can be an organic linker. For example, the DNA for the long arm or the short arm can be an organic linker.

The four streptavidin subunits are related by 222 point group symmetry. The biotin carboxyl group resides at the surface of the protein and is the point of attachment of all biotin-conjugates. The hydroxy oxygen atom (of the carboxyl group) on each bound biotin molecule form a pseudo-rectangular plane with dimensions of (3.11×1.87)nm and a dihedral angle of 25.20°.

A linker/biotin composition can include at least one linker. For example, at least one linker can connect two biotin molecules such that the resulting composition can bind to at least two biotin binding sites of a streptavidin, thereby blocking two binding sites.

A linker/biotin composition can include at least two linkers. For example, a first linker can connect a first biotin and a second biotin and a second linker can connect the second biotin and a third biotin such that the resulting composition can bind to at least three biotin binding sites of a streptavidin, thereby blocking three binding sites. As another example, a first linker can connect a first biotin and a second biotin and a second linker can connect the first linker and a third biotin such that the resulting composition can bind to at least three biotin binding sites of a streptavidin, thereby blocking three binding sites. Where a second linker attaches to a first linker, the second linker can be attached anywhere along the length of the first linker such that third biotin can bind to a biotin binding site of a streptavidin. For example, a second linker can attach to a first linker so as to form a “T” or a “Y” configuration. As another example, a second linker can attach to a first linker so as to form an “L” configuration.

The generally rectangular orientation of a streptavidin results in a “long” side and a “short” side between different biotin binding sites. Thus a length of a linker can be configured based upon the different side lengths of a streptavidin. For example, a linker/biotin composition can be configured to include one short linker and one long linker connecting three biotins such that the resulting composition can bind to at least three biotin binding sites of a streptavidin, thereby blocking three binding sites.

Two bound biotin ligands on the shorter side can be linked together in bidentate fashion by linkers as short as about 1.89 nm (i.e., a “dual biotin”). For example, a linker for linking two biotins so as to bind to two biotin binding sites on the “short” side of a streptavidin can be at least about 1.8 nm, at least about 1.9 nm, at least about 2.0 nm, at least about 2.5 nm, at least about 2.6 nm, at least about 2.7 nm, at least about 2.8 nm, at least about 2.9 nm, or longer. A “short” linker can be as long as desired so long as it permits binding of two biotins to “short” side streptavidin binding sites and does not substantially interfere with binding of another biotin to a “long” side streptavidin binding site.

The longer side of streptavidin is 3.11 nm, but this distance is directly through the protein and therefore linkers to connect two long side bound biotins can be greater than 6.0 nm, the distance “around” the protein. For example, “long” side biotin linker can be at least about 6 nm, at least about 7 nm, at least about 8 nm, at least about 9 nm, at least about 10 nm, at least about 11 nm, at least about 12 nm, at least about 13 nm, at least about 14 nm, at least about 15 nm, at least about 16 nm, at least about 17 nm, at least about 18 nm, at least about 19 nm, at least about 20 nm, or more. A “long” linker can be as long as desired so long as it permits binding of a biotin to a “long” side streptavidin binding site and does not substantially interfere with binding of one or two biotins to a “short” side streptavidin binding site(s). In some embodiments, the tridentate biotin ligands are about 14 nanometers stretched. When the oligonucleotide (e.g., a 14 nanometer oligonucleotide) is hybridized to it's complement, it can be shortened by almost half it's length. Further, DNA duplexes have a persistence length of 45 nanometers. As such, severe strain would occur if the duplex was able to bridge two “long-side” biotins.

A linker can include a nucleic acid. Where a linker comprises a nucleic acid, exposure of the composition to a complementary nucleic acid can result in binding of the complementary sequences, straightening, partial straightening, or substantial straightening of the linker sufficient to dislodge a linked biotin from a biotin binding site of a streptavidin (see e.g., FIG. 15). In such configuration, straightening of the tether can provide enough force to unbind the linked biotin from streptavidin.

Methods

A streptavidin having one or more biotin sites blocked (e.g., a monovalent streptavidin) described herein can be used in a variety of biotin-related methods and assays, including but not limited to purification (e.g., affinity chromotography) or detection (e.g., tagged detection strategies using enzyme reporters or fluorescent probes). Localization can be according to fluorescent or electron microscopy, ELISA assays, ELISPOT assays, western blots and other immunoanalytical methods. Detection with a monovalent streptavidin can avoid clustering or aggregation of the biotinylated target. Conventional streptavidin-biotin protocols are well developed in the art and such protocols can be adapted to employ a streptavidin having one or more biotin sites blocked.

A streptavidin having one or more biotin sites blocked can bind to a biotinylated target molecule. A biotinylated target molecule can be, for example, a protein, nucleic acid or other molecule or substrate.

Biotinylation is the process of covalently attaching a biotin to a molecule or substrate. Biotinylation is generally rapid, specific and is unlikely to perturb the natural function of the molecule or substrate to which it is attached given the small size of a biotin (e.g., MW=244.31 g/mol). Biotin can bind to streptavidin with an extremely high affinity, fast on-rate, and high specificity, and these interactions can be exploited as described herein. Biotin-binding to streptavidin can be resistant to extremes of heat, pH, or proteolysis, which can allow use of a biotinylated molecule or substrate in a wide variety of environments. Furthermore, multiple biotin molecules can be conjugated to a molecule or substrate, which can allow binding of multiple streptavidin. A large number of biotinylation reagents are know in the art and commercially available.

Biotinylation of a target molecule can be according to conventional means, such as enzymatic biotinylation, primary amine biotinylation, sulfhydryl biotinylation, carboxyl biotinylation, oligonucleotide biotinylation, or non-specific biotinylation. For example, chemical biotinylation uses conjugation chemistries to yield nonspecific biotinylation of amines, carboxylates, sulfhydryls or carbohydrates (e.g., NHS-coupling gives biotinylation of any primary amines in the protein). As another example, enzymatic biotinylation can result in biotinylation of a specific lysine within a certain sequence by a bacterial biotin ligase. A biotinylation reagent can include a reactive group attached via a linker to the valeric acid side chain of biotin. As the biotin binding pocket of streptavidin is buried beneath the protein surface, biotinylation reagents possessing a longer linker can be desirable, as they enable the biotin molecule to be more accessible to binding streptavidin protein. This linker can also mediate the solubility of biotinylation reagents. For example, biotinylation linkers that incorporate poly(ethylene) glycol (PEG) can make water-insoluble reagents soluble or increase the solubility of biotinylation reagents that are already soluble to some extent.

Primary Amine Biotinylation.

Biotin can be conjugated to an amine group on the molecule or substrate. A primary amine group can be present as a lysine side chain epsilon-amine or N-terminal α-amine. Amine-reactive biotinylation reagents can be divided into two groups based on water solubility.

N-hydroxysuccinimide (NHS) esters have poor solubility in aqueous solutions. For reactions in aqueous solution, NHS can be first be dissolved in an organic solvent, then diluted into the aqueous reaction mixture. Commonly used organic solvents for this purpose can include dimethyl sulfoxide (DMSO) and dimethyl formamide (DMF). Because of the hydrophobicity of NHS-esters, NHS biotinylation reagents can also diffuse through the cell membrane, meaning that they will biotinylate both internal and external components of a cell.

Sulfo-NHS esters are more soluble in water and can be dissolved in water just before use because they hydrolyze easily. The water solubility of sulfo-NHS-esters is due at least in part from a sulfonate group on the N-hydroxysuccinimide ring. Water solubility can eliminate a need to dissolve the reagent in an organic solvent. Sulfo-NHS-esters of biotin do not penetrate the cell membrane.

The chemical reactions of NHS- and sulfo-NHS esters can be identical, in that they can both react spontaneously with amines to form an amide bond. Because the target for the ester is a deprotonated primary amine, the reaction is favored under basic conditions (above pH 7). Hydrolysis of the NHS ester is a major competing reaction, and the rate of hydrolysis increases with increasing pH. NHS- and sulfo-NHS-esters have a half-life of several hours at pH 7 but only a few minutes at pH 9.

There is additional flexibility in the conditions for conjugating NHS-esters to primary amines. Incubation temperatures can range from about 4-37° C., pH values in the reaction range from about 7-9, or incubation times range from a few minutes to about 12 hours. Buffers containing amines (e.g., Tris or glycine) can be avoided, because they compete with the reaction.

Sulfhydryl Biotinylation.

An alternative to primary amine biotinylation is to label sulfhydryl groups with biotin. Sulfhydryl-reactive groups such as maleimides, haloacetyls, or pyridyl disulfides, can require free sulfhydryl groups for conjugation; disulfide bonds can be first reduced to free up the sulfhydryl groups for biotinylation. If no free sulfhydryl groups are available, lysines can be modified with various thiolation reagents (Traut's Reagent, SAT(PEG4), SATA and SATP), resulting in the addition of a free sulfhydryl. Sulfhydryl biotinylation can be performed at a slightly lower pH (e.g., about 6.5-7.5) than labeling with NHS esters.

Carboxyl Biotinylation.

Biotinylation reagents that target carboxyl groups do not have a carboxyl-reactive moiety per se but instead rely on a carbodiimide crosslinker such as EDC to bind the primary amine on a biotinylation reagent to a carboxyl group on the target.

Biotinylation at carboxyl groups can occur at a pH of about 4.5-5.5. To prevent crossreactivity of the crosslinker with buffer constituents, buffers should not contain primary amines (e.g., Tris, glycine) or carboxyls (e.g., acetate, citrate).

Glycoprotein Biotinylation.

Glycoproteins can be biotinylated by modifying the carbohydrate residues to aldehydes, which can then react with hydrazine- or alkoxyamine-based biotinylation reagents. Sodium periodate can oxidize a sialic acid on glycoproteins to aldehydes to form these stable linkages at a pH of about 4-6.

Antibodies can be heavily glycosylated, and because glycosylation does not interfere with the antibody activity, biotinylating the glycosyl groups can be an ideal strategy to generate biotinylated antibodies.

Biotinylation at carboxyl groups can occur at a pH of about 4.5-5.5. To prevent crossreactivity of the crosslinker with buffer constituents, buffers should not contain primary amines (e.g., Tris, glycine) or carboxyls (e.g., acetate, citrate).

Oligonucleotide Biotinylation.

Oligonucleotides can be readily biotinylated in the course of oligonucleotide synthesis by the phosphoramidite method using, e.g., commercial biotin phosphoramidite. Upon the standard deprotection, the conjugates obtained can be purified using reverse-phase or anion-exchange HPLC.

Non-Specific Biotinylation.

Photoactivatable biotinylation reagents can be useful when primary amines, sulfhydryls, carboxyls or carbohydrates are not available or not desired for labeling. A photoactivatable biotinylation reagent relies on aryl azides, which become activated by ultraviolet light (UV; >350 nm), which then react at C—H and N—H bonds. A photoactivatable biotinylation reagent can also be used to activate biotinylation at specific times by simply exposing the reaction to UV light at the specific time or condition.

Imaging

A streptavidin having one or more biotin sites blocked (e.g., a monovalent streptavidin) described herein can be used for imaging, such as live cell imaging. Fluorescently labeled streptavidin complex can be used to label cell surfaces according to, for example, experiments described in Howarth and Ting 2008 Nature Protocols 3(3), 534, or Howart et al. 2006 Mature Methods 3(4) 267-273, both directed to labeling using genetically engineered monovalent streptavidin.

A conventional approach genetically mutates three of streptavidin's four binding sites thereby block binding in the three mutated binding sites (see e.g., Howarth and Ting 2008 Nature Protocols 3(3), 534, or Howart et al. 2006 Nature Methods 3(4) 267-273). In contrast, a monovalent streptavidin described herein can use a trivalent biotin ligand to block three of streptavidin's four biotin binding sites. The monovalent streptavidin compositions and protocols described herein are significantly less complex than existing approaches.

Costs associated with monovalent streptavidin compositions and protocols described herein in terms of, for example, time, complexity, or difficulty of the procedure, can be at least an order of magnitude less. Further, monovalent streptavidin compositions and protocols described herein provide flexibility to add additional functionalities to streptavidin, such as fluorescent labels, etc., during formation of the linker.

A “monovalent streptavidin” can be used in cell imaging. Despite streptavidin being invaluable in imaging applications, the ability of streptavidin to naturally bind four biotins without discrimination can be a drawback under certain circumstances. For example, such indiscriminate binding can lead to problems due to crosslinking when labeling cellular components in living cells, as unwanted crosslinking can result in the function of the target under investigation becoming altered or destroyed.

Oligonucleotide Detection

A monovalent streptavidin described herein can be used for analysis or detection of oligonucleotide sequences. Choosing an appropriate linker can make a monovalent streptavidin specific for a particular complementary short oligonucleotide (see e.g., FIG. 15). An oligonucleotide sequence can be, for example, a miRNA. Such a method can obviate the need for problematic sandwich detection of short oligonucleotides, which can form unstable tripartite complexes.

No known technologies use the binding event of a target oligonucleotide (e.g. microRNA) to a streptavidin-oligonucleotide (probe) conjugate to expose a streptavidin bound biotin moiety which can then be visualized by labeling. Conventional approaches for oligonucleotide sample analysis are multi-step procedures more involved or complex than methods described herein.

Using a monovalent streptavidin composition as a probe for target oligonucleotides, such as microRNA, and associated protocols as described herein can dramatically decrease the number of steps involved, thereby speeding up time of analysis and providing ease of use.

A tridentate-biotin ligand can be complexed to streptavidin, resulting in an assembly with single mismatch sensitivity that can determine if biotin is exposed or not. In some embodiments, if a target oligonucleotide is fully matched to the tridentate-biotin ligand, a biotin is exposed; if a single mismatch is present, the biotin is not exposed or exposed at a slower rate.

MicroRNAs can be short nucleotide sequences. For example, a MicroRNA can be about 22 nucleotides in length. For example, a plurality of MicroRNAs can have an average length of about 22 nucleotides. As another example, a MicroRNA can be about 15 to about 35 nucleotides in length (e.g., about 15, about 16, about 17, about 18, about 19, about 20, about 21, about 22, about 23, about 24, about 25, about 26, about 27, about 28, about 29, about 30, about 31, about 32, about 33, about 34, or about 35 nucleotides in length). As another example, a MicroRNA can be about 15 to about 30 nucleotides in length. As another example, a MicroRNA can be about 20 to about 25 nucleotides in length. As another example, a MicroRNA can be about 20, about 21, about 22, about 23, about 24, or about 25 nucleotides in length.

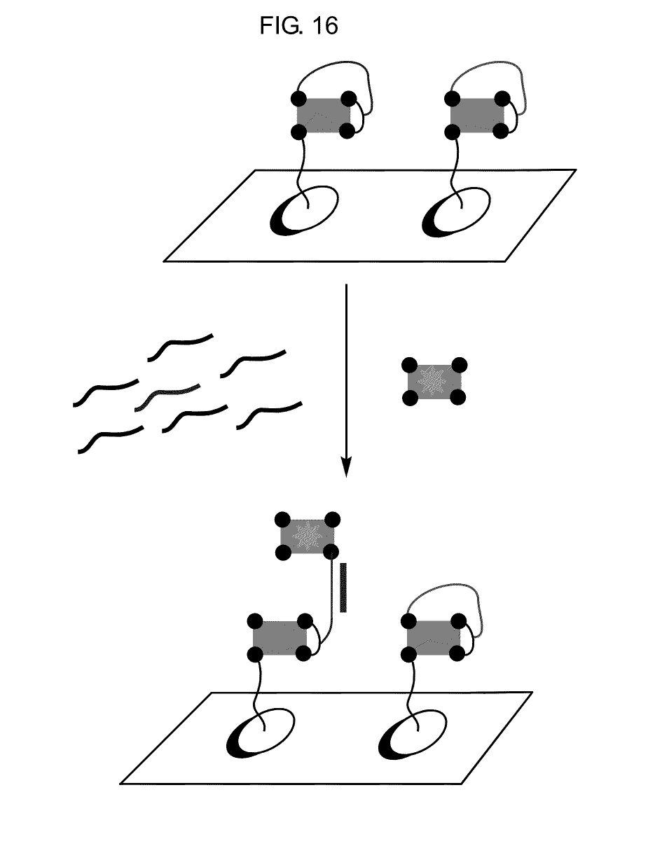

In some embodiments, a method of microRNA detection can be as illustrated in FIG. 16.

Determination of Number of Attached Biotin

One aspect of the present disclosure provides a method for determining a number of biotins attached to a target (e.g., a protein) after carrying out a biotinylation reaction on a protein. This method can employ a reagent added to a small amount of biotinylated protein sample (e.g., about 10 picomoles) and analyzed by standard polyacrylamide gel electophoresis (PAGE). In contrast to conventional systems, the method does not require a fluorescence spectrometer for analysis of sub-microgram amounts of protein (as used for the HABA/avidin system). Various embodiments require only an inexpensive electrophoresis setup. While the HABA/avidin system can also be read using a UV-vis spectrometer, such method requires, e.g., over 1000 times the amount of protein than used in a method described herein.

A monovalent streptavidin described herein can be used to quantify an extent of biotinylation of a target, such as a protein. A biotinylation reaction with a target can be as described herein. A sample of the biotinylated target can be mixed with monovalent streptavidin so as to “tag” accessible biotins of the target. The tagged sample (i.e., a biotinylated target complexed with monovalent streptavidin) can then be analyzed to determine, e.g., quantity of biotinylation.

An amount of monovalent streptavidin to be mixed with a sample of the biotinylated target can be about 1:1, about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, about 1:20, or more. One of ordinary skill will understand that monovalent streptavidin can be added in excess of the number of biotins present in the sample so as to ensure sufficient tagging. An amount of monovalent streptavidin to be mixed with a sample of the biotinylated target can be adjusted with respect to the amount of biotinylation expected, calculated, or observed.

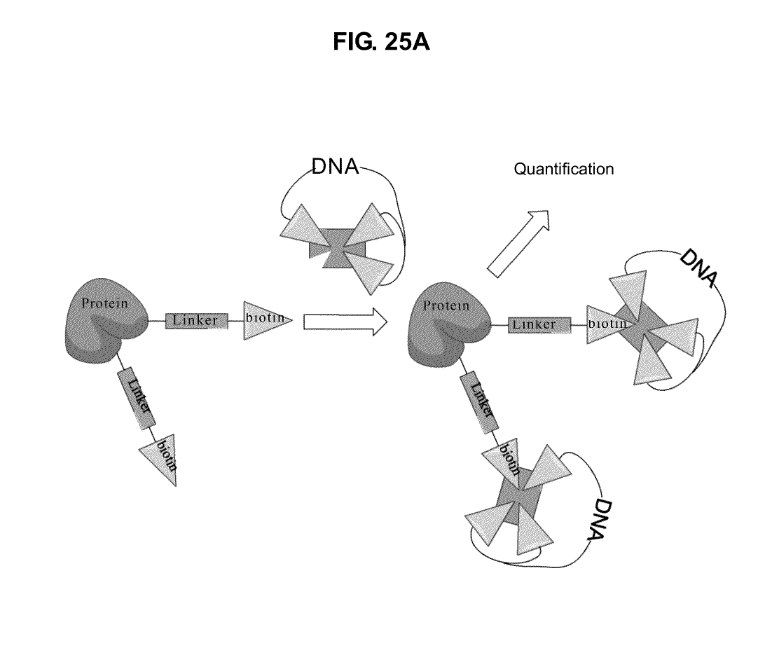

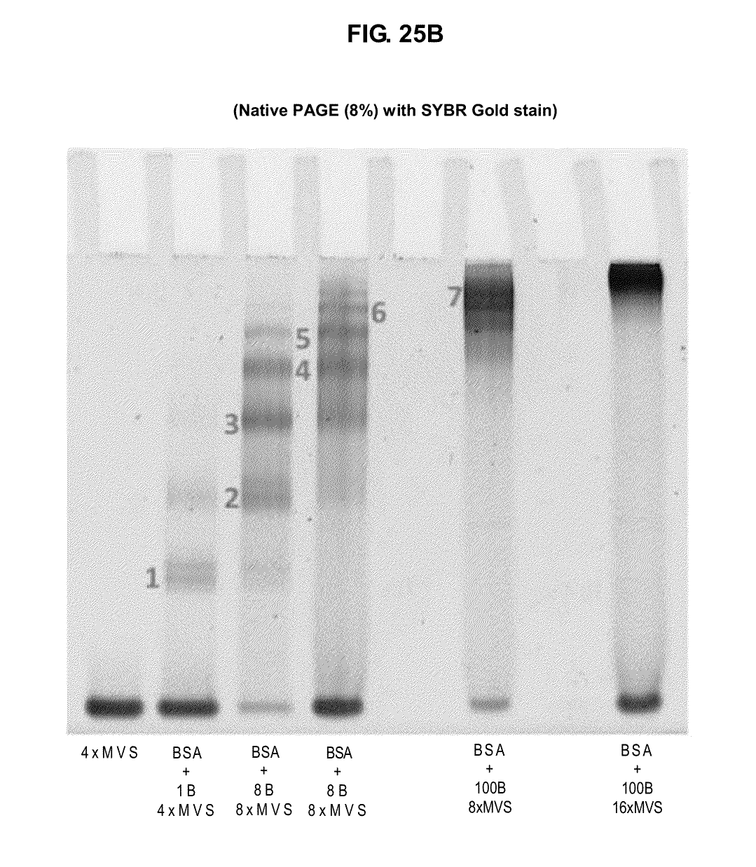

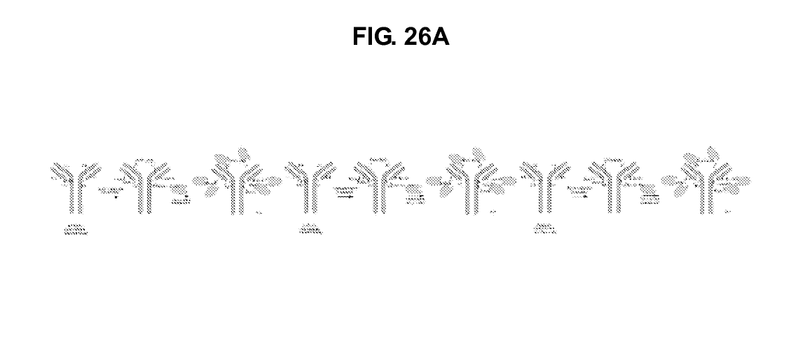

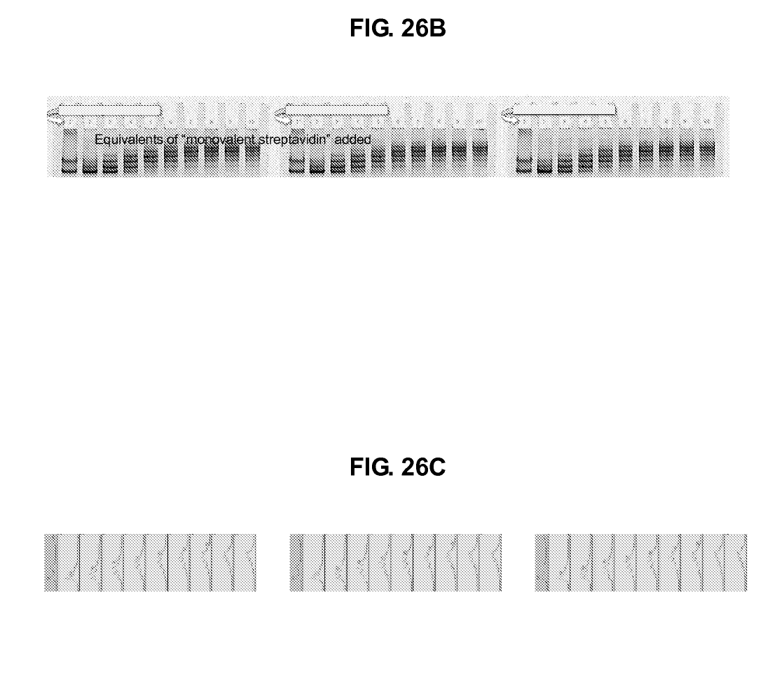

In some embodiments, a series of samples comprising different amounts of monovalent streptavidin are analyzed. For example, a plurality of samples of biotinylated target (e.g., about 10 pM) can be combined with monovalent streptavidin in amounts of about 1:1, about 1:2, about 1:3, about 1:4, about 1:5, about 1:6, about 1:7, about 1:8, about 1:9, or about 1:10 (see e.g., Example 13; FIG. 26B). Such an array of increasing amounts of monovalent streptavidin can provide information regarding the distribution of biotins per target (see e.g., FIG. 25B; FIG. 26B; FIG. 26C).

The sample of the biotinylated target used for quantification can be relatively small compared to conventional quantification assays. For example, the sample of the biotinylated target used for quantification can be less than about 100 pM. As another example, the sample of the biotinylated target used for quantification can be less than about 90 pM, less than about 80 pM, less than about 70 pM, less than about 60 pM, less than about 50 pM, less than about 40 pM, less than about 30 pM, less than about 20 pM, or less than about 10 pM. As another example, the sample of the biotinylated target used for quantification can be about 1 pM up to about 10 pM, or about 1 pM up to about 50 pM, or about 1 pM up to about 100 pM.

There is not necessarily an upper functional limit of the sample size. But one of ordinary skill will understand that a small sample of biotinylated target can provide an advantage of conserving such biotinylated target for its intended use, rather than unnecessarily using excess levels of sample for quantifiation. Nonetheless, a quantification method described herein can be used with larger sample. For example, the sample of the biotinylated target used for quantification can be at least about 1 pM, at least about 10 pM, at least about 20 pM, at least about 30 pM, at least about 40 pM, at least about 50 pM, at least about 60 pM, at least about 70 pM, at least about 80 pM, at least about 90 pM, or at least about 100 pM.

Analysis of the tagged sample (i.e., a biotinylated target complexed with monovalent streptavidin) can be according to any conventional technique used to separate or quantify (absolutely or relatively) biological macromolecules. Analytical techniques for macromolecules are well understood in the art and conventional assays can be adapted for use according to methods described herein. For example, analysis of the tagged sample (i.e., a biotinylated target complexed with monovalent streptavidin) can be according to an electrophoresis technique. As another example, analysis of the tagged sample (i.e., a biotinylated target complexed with monovalent streptavidin) can be according to gel electrophoresis. As another example, analysis of the tagged sample (i.e., a biotinylated target complexed with monovalent streptavidin) can be according to polyacrylamide gel electrophoresis (PAGE), or variant techniques thereof (E.g., SDS-PAGE). As another example, analysis of the tagged sample (i.e., a biotinylated target complexed with monovalent streptavidin) can be according to capillary gel electrophoresis (CGE), capillary isoelectric focusing (CIEF), capillary isotachophoresis and micellar electrokinetic capillary chromatography (MECC). As another example, analysis of the tagged sample (i.e., a biotinylated target complexed with monovalent streptavidin) can be according to densitrometry analysis (e.g., in conjucntion with an electrophoretic separation technique).

Furthermore, a method described herein can show not only show the degree of biotinylation achieved, but, unlike conventional methods, can also show the distribution of products obtained (i.e., an actual spread of reaction products as opposed to conventional methods providing an average). A spread of reaction products can be expressed as percentage of biotinylated targets having one biotin, two biotins, three biotins, etc. For example, an HABA/avidin system may report an average 5 biotins per protein (i.e. a 5:1 ratio), but the present method can report an actual spread. An exemplary result may be, for example, 3.4% at 8:1, 8.6% at 7:1, 24% at 6:1, 23.6% at 5:1, 18.7% at 4:1, 13.5% at 2:1 and 2.4% at 1:1. These example illustrates a format of hypothetical results.

Molecular Engineering

Design, generation, and testing of the variant nucleotides, and their encoded polypeptides, having the above required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art. Generally, conservative substitutions can be made at any position so long as the required activity is retained.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41(fraction G/C content)−0.63(% formamide)−(600/l). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides, protein aptamers, nucelotide aptamers, and RNA interference (RNAi) (e.g., small interfering RNAs (sRNA), short hairpin RNA (shRNA), and micro RNAs (miRNA) (see e.g., Fanning and Symonds (2006) Handb Exp Pharmacol. 173, 289-303G, describing hammerhead ribozymes and small hairpin RNA; Helene, C., et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several sRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinofrmatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to a streptavidin having two or more biotin binding sites blocked by tethered biotin molecules, as described herein. The streptavidin and tethered biotin molecules can be provided together or separately. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

### METHODOLOGY

The following example provides methodology for assembly of monovalent streptavidin, PAGFE, HPLC purification, and UV-vis characterization.

Biotinylated oligonucleotides are:

(SEQ ID NO: 13)
(1) = 5-/52-Bio/TTT TTT TTT TTT TTT TTT TTT TTT T-
/3BioTEG/-3
(SEQ ID NO: 14)
(2) = 5-/52-Bio/GAC TAT CGC CTT CAT ACT ACC TCC-
/3BioTEG/-3
(SEQ ID NO: 15)
(3) = 5-/52-Bio/GAC TAT CGC CTT CAT ACT AC
/3BioTEG/-3
(SEQ ID NO: 16)
(4) = 5-/52-Bio/GAC TAT CGC CTT CAT ACT ACC TCC-
/iBiodT//3Bio/-3

Abbreviations: 52-Bio is a 5′-end dual-biotin modification; 3BioTEG and 3Bio are 3′-end monobiotin modifications; iBiodT is a biotin functionalized thymine nucleotide.

All experiments were carried out at room temperature unless stated otherwise. All oligonucleotides were commercially manufactured by Integrated DNA Technologies Inc. (Coralville, Iowa).

ASSEMBLY: In general, tris-biotinylated oligonucleotide (250 microL×1 microM) is mixed as quickly and as evenly as possible with streptavidin (250 microL×1 microM) at room temperature. The resulting mixture is purified by anion exchange HPLC. Further details are as follows.

Assembly of STV•5′-(biotin)₂-oligonulceotide-biotin-3′ conjugates: The commercially supplied lyophilized (biotin)₂-oligonulceotide-biotin-3′ was resuspended in distilled, deionized water (Mediatech, Cat. No. 46-000-CM, Lot No. 46000046) to give a stock solution concentration of 100 microM. Commercially supplied lyophilized streptavidin (ThermoFisher, Cat. No. 21135, Lot. No. LI150275) was resuspended to give a stock solution concentration of 10 mg/mL. (biotin)₂-oligonulceotide-biotin-3′ stock solution (2.5 microL, 0.25 nmol) was added to buffer (250 microL of 20 mM TRIS, pH 7.2, 150 mM NaCl). Streptavidin stock solution (1.325 microL, 0.25 nmol) was added to buffer (250 microL of 20 mM TRIS, pH 7.2, 150 mM NaCl). The buffered (biotin)₂-oligonulceotide-biotin-3′ solution was distributed as evenly and quickly as possible into the diluted streptavidin solution (250 microL). To maximize the yield of the desired monovalent streptavidin product, the solution was heated in a water bath at 70° C. for 15 minutes then stood to cool to room temperature on the bench. The same procedure can also be carried out in the presence of 0.5 mM desthiobiotin for increased yield without heating, however the reaction needs a period of three days to reach equilibrium. {F-STV-(3)}.

PAGE: The complement strand was resuspended in distilled, deionized water to give a stock solution concentration of 100 microM. The target strand (0.2 microL, 20 pmol) was added to the purified monovalent streptavidin complex solution (0.43 microM, 23 microL, 10 pmol) and incubated at room temperature for 15 mins. Loading dye (2 microL, TRIS 160 mM, glycerol 20%, bromophenolblue 0.04%) was added and the sample loaded on to a gel composed of a 4% polyacrylamide stacking layer and 10% polyacrylamide separation layer. Native gels were run in TRIS-Glycine buffer at 100V through the 4% stacking layer then at 200V through the 10% seperation layer.

HPLC Purification: The instrument used was a Shimadzu Prominence system. The complexes were purified on an anion exchange column: Waters BioSuite Q 10 μm AXC 7.5×75 mm column (Part No. 186002177, Lo No. 081M192231). For example, for STV-(2), a gradient of 22-to-34% “B” in “A” over 19.9 mins was used eluting the desired product at 9.2-to-11.2 mins (where “A” is TRIS 20 mM, pH 7.2, and “B” is TRIS 20 mM, pH 7.2, with NaCl 1 M). Flow rate was 1 mLmin⁻¹; or on a TSKgeI DEAE-NPR column, 4.6×50 mm (ID×L), (TOSOH BIOSCIENCES). Purified complexes were concentrated (if desired) using Amicon Ultra Centrifugal Filter Devices (Millipore, Regenerated Cellulose 30,000 MWCO).

UV-vis A₂₆₀ₙₘ/A₂₈₀ₙₘ characterization of STV-(2): STV-(2) has a DNA/protein (260 nm/280 nm) absorbance ratio of 1.05. The 1.05 ratio can be compared to the standards STV-(GAC TAT CGC CTT CAT ACT ACC TCC-monobiotin-3′)ₙ (SEQ ID NO: 14), where n=1-4^{[Pei 2006]} and also compared to the crosslinked streptavidin dimer [STV-(2)-STV] i.e. STV and (2) in a ratio of 2:1, which have respective ratios of 1.03, 1.24, 1.36, 1.43, and 0.87, supporting that STV-(2) is composed of STV and (2) in a 1-to-1 ratio. The STV-(2) product has closer anion exchange HPLC elution properties to the conjugate STV-(GAC TAT CGC CTT CAT ACT ACC TCC-monobiotin-3′)₁ (SEQ ID NO: 14) (Figure S3-i), supporting the formulation of a discrete STV-(2) conjugate as opposed to a [-STV-(2)-]ₙ oligomer. The monovalent nature of STV-(2) (Figure S3-v) was shown by titrating in the mono-biotinylated oligonucleotide GAC TAT CGC CTT CAT ACT ACC TCC-monobiotin-3′ (SEQ ID NO: 14), which demonstrated that STV-(2) binds to only one B₁ (Figure S3 vi-viii). UV absorbance was determined on an Amersham Biosciences Ultrospec 3300 pro UV/visible spectrophotometer.

ELISA: All steps were performed at room temperature. A 96-well Streptavidin Coated White Plate (Thermo Scientific Prod #15218) was washed six times using a squirt bottle of PBS and then 100 mciroL of PBS added to each well. The biotinylated oligonucleotide/5BioTEG/CGGTTTTTTGTTCTTTGTTTTGTTCTTTGC (5) (SEQ ID NO: 17) (0.5 microL of 10 microM) was then added and incubated for 7 mins. The mixture was removed by pippette and washed once with PBS by pipette. The wells were than washed six times using a squirt bottle of PBS and then 100 microL of PBS added to each well./5BioTEG/GCAAAGAACAAAACAAAGAACAAAAAACCG (6) (SEQ ID NO: 18) (0.5 microL of 10 uM) or STV-(3)•(6) (5.6 microL—made by adding 2 uL of 1 microM (6) to 54 microL of 369 nM monoval STV-(3) and incubating for 5 mins) was then added to respective wells and incubated for 37 mins. The mixture was removed by pipette and washed once with PBS by pipette. The wells were than washed six times using a squirt bottle of PBS and then 100 microL of PBS added to each well. 17 mer ‘target’ (perfect match or single CC mismatch) (5 microL of 100 microM) was added and incubated for 15 mins. The mixture was removed by pipette but wells were not washed. HRP-streptavidin conjugate (100 microL of 1/40,000 diluted stock solution—Thermo Scientific Prod #21130 was then added and incubated for 5 minutes. The mixture was removed by pippette and washed once with PBS by pipette. The wells were than washed six times using a squirt bottle of PBS with upside down ‘banging’ of the plate on a hard surface (covered with a absorbant) to ensure all traces of unbound HRP-STV are removed from the wells. Lastly, substrate solution (100 microL—Thermo Scientific Prod #15159) was added to each well.

### Example 2

### Testing of Streptavidin-Tridentate Biotin

The following example describes testing of streptavidin-tridentate biotin. Methods are according to Example 1 unless described otherwise.

Monovalent streptavidin was constructed via a direct macrocyclization reaction with a tris-biotinylated oligonucleotide; the oligonucleotide was designed with dimensions that enabled it to intramolecularly bind to streptavidin using all three biotin moities; this is in contrast to previously reported designs, in which intramolecular cyclization was disfavored by using very short tris-biotinylated ligands.

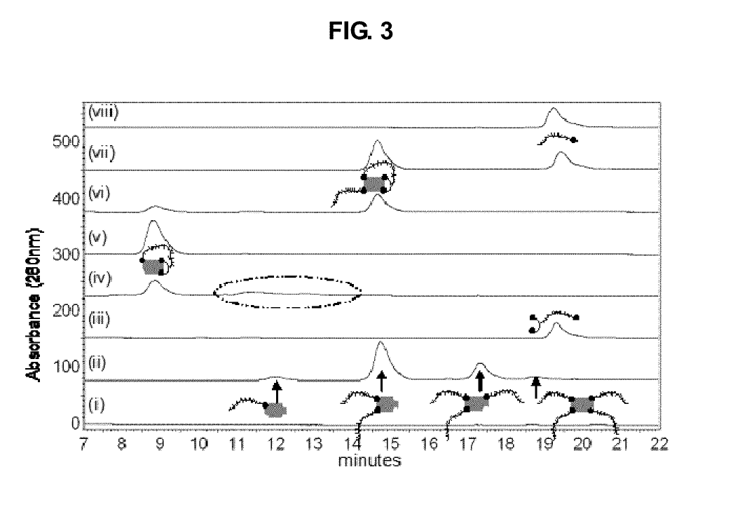

Tris-biotinylated oligonucleotide 5′-(biotin)₂-T₂₅-biotin-3′ (1) (SEQ ID NO: 13) was tested. Mixing of this oligonucleotide with one equivalent of streptavidin (STV) resulted in an estimated yield of 50% for the major product monovalent streptavidin STV-(1) (yields determined by HPLC peak area integration). The monovalency of the conjugate was confirmed by the ability of STV-(1) to accept only one additional biotinylated oligonucleotide (see e.g., FIG. 1B, lanes 2-4; FIG. 3).

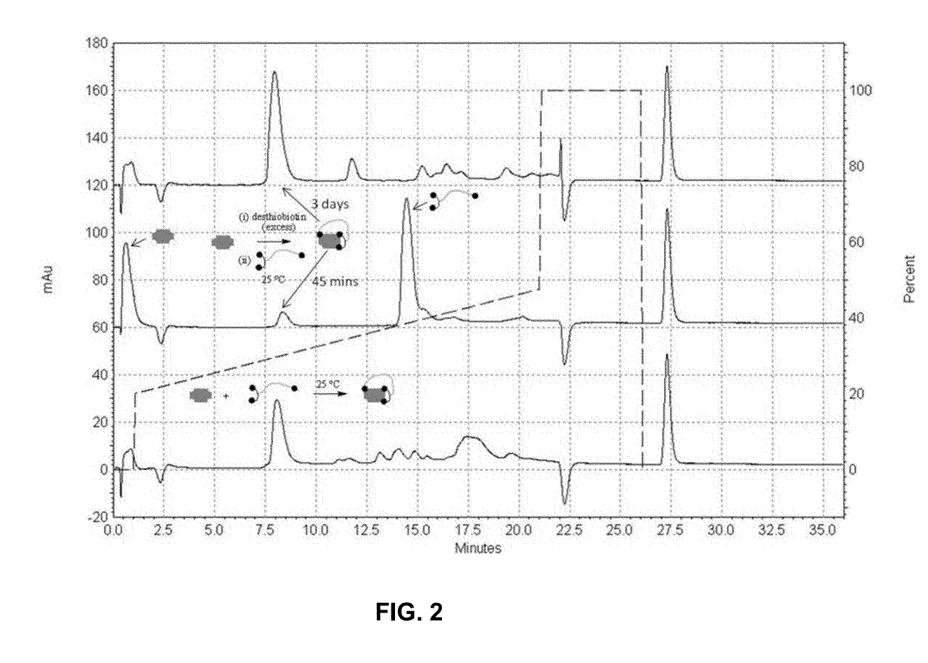

Analogous results were obtained when the T₂₅ sequence was substituted with an arbitrarily chosen 24- or 20-mer nucleotide sequence, STV-(2) and STV-(3), respectively. Yields decreased with shortening of the oligonucleotide; for example, STV-(2) gave an estimated yield of 30%. But this yield could be increased to 70% using two equivalents of streptavidin and heating to 70° C. then cooling. Without heating, the yield was also increased to 50% by adding the oligonucleotide to streptavidin in the presence of an excess of desthiobiotin (K_{d}˜10⁻¹⁰)—presumably the slower biotin-streptavidin binding (days for equilibrium to be established rather than milliseconds) removes varying local effective concentrations that are encountered when trying to mix reagents to create a homogenous mixture on a millisecond time scale (see e.g., HPLC traces in FIG. 2 and FIG. 3). On its own, the highest yield of 70% is a significant improvement as far as generation of monovalent streptavidin goes when compared to statistically generated three-legged “spiders” (Pei et al. 2006 J. Am. Chem. Soc. 128, 12693-12699) and modified streptavidin incorporating non-binding monomers at 35% (statistical theoretical maximium is 42%) (Howarth et al. 2006 Nat. Methods 3, 267-273). Whilst the oligonucleotide linker could be substituted with any other moiety of sufficient length and flexibility, it had an important benefit—straightforward isolation of the desired product via anion exchange HPLC. The yield, time, and ease of this procedure to produce a single vacant biotin binding site are a significant improvement over the approach based on protein engineering (cf. Howarth et al. 2006 Nat. Methods 3, 267-273).

### Example 3

### STV-B3

STV-B3 was prepared by combining rapidly 1 microMolar streptavidin (STV) and 1 microMolar 5′-dualbiotin-T25-monobiotin-3′ (B3) in equimolar amounts at 25° C., a one-to-one STV-B3 complex (c.a. 50% yield by HPLC chromatograph major peak) was obtained.

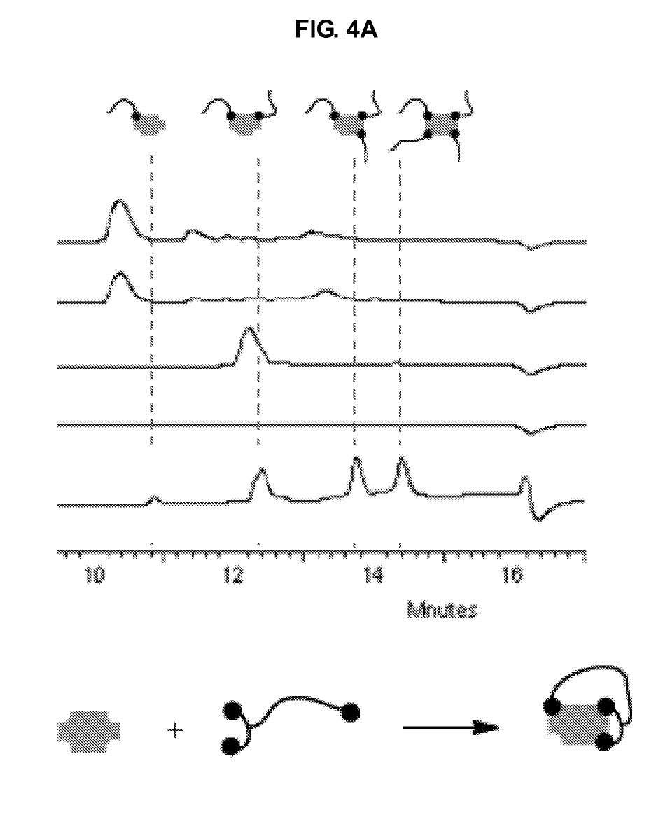

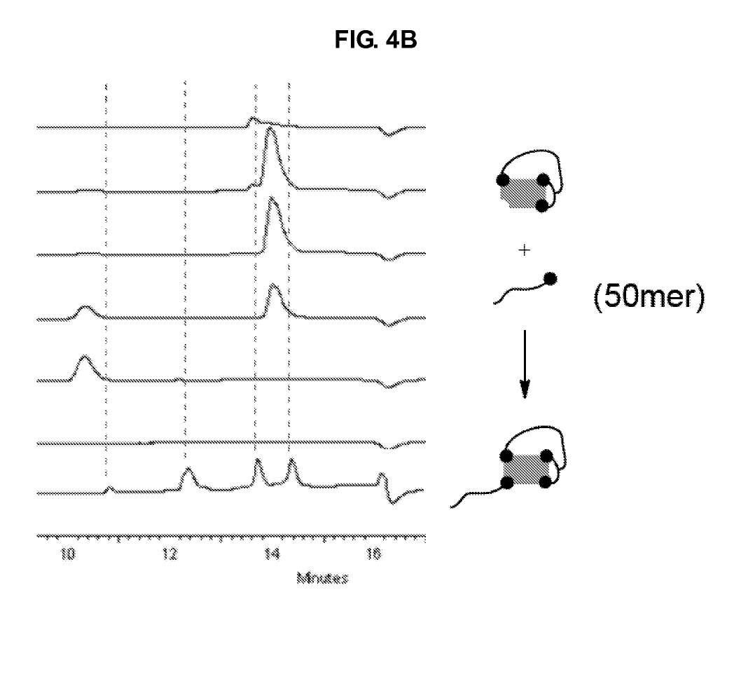

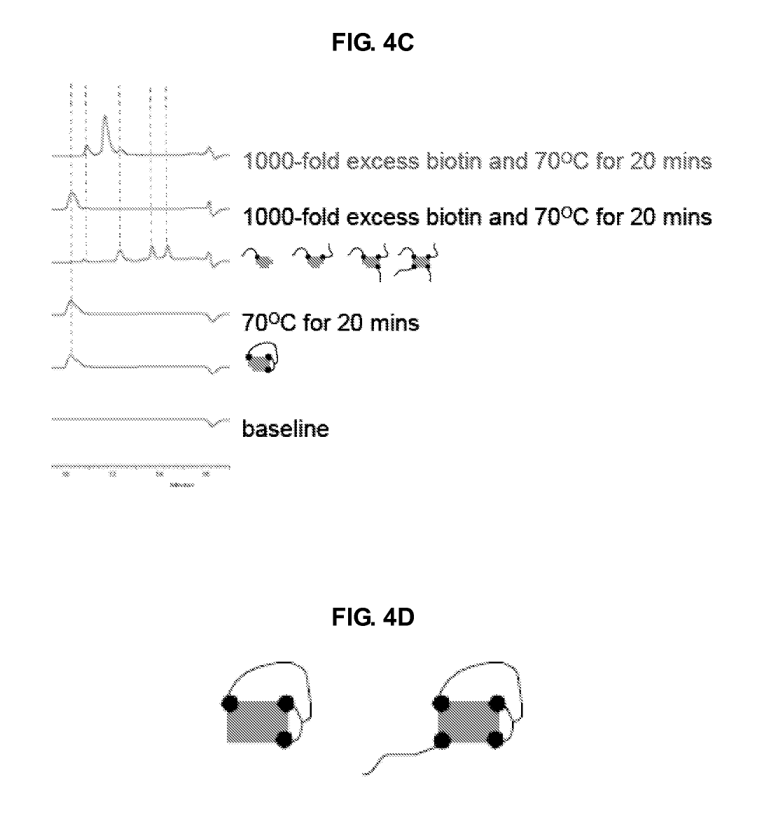

Results showed that the major product (STV-B3) has similar properties to STV-5′-(monobiotin-T25)1 and not STV-(5′-biotin-T25)n, where n is 2, 3, or 4, supporting the assignment of a discrete 1-to-1 complex (see e.g., FIG. 4A). The monovalent nature of the product was shown by titration of pure STV-B3 with a fifty base oligonucleotide 5′-monobiotin-50 mer (see e.g., FIG. 4B). Identification of species formed was made from IE-HPLC trace titrations and comparison to known standards, along with UV-vis data, such as 260 nm/280 nm to qualify DNA/protein ratio.

These results support that all biotins in the complex are bound to the same STV, which yields a monovalent streptavidin species.

With respect to stability, the purified product showed no change over several months at 4° C. In addition, STV-B3 was stable in extreme conditions of 70° C. with 1000-fold excess of biotin present (see e.g., FIG. 4C). In contrast, a monobiotin-oligonucleotide is easily displaced from streptavidin under these same conditions (see e.g., FIG. 4C). In addition, even at 10-fold excess STV to B3, the predominant product is still STV-B3, demonstrating the preference and stability of this ligand for a 1:1 complex.

When streptavidin (STV) and B3 are combined in equimolar amounts, (taking into full account the molecular dimensions of STV and B3) only the complexes depicted in FIG. 4D are possible. Experimentally (at 25° C.), a 1-to-1 STV-B3 complex (>x %) was obtained, as evidenced by the anion-IE-HPLC trace of the reaction mixture (compared to a STV-(T25)n ladder, where n=1-to-4), and HPLC traces of the titration of the purified product with sub and excess amounts of biotinylated oligo (B1) to give B1-STV-B3 exclusively.

The purified product showed no change over several months at 4° C. Therefore, evidence supports all biotins in the complex are bound to the same STV, which yields the “monovalent streptavidin” species.

### Example 4

### Hybridization Behavior of the Oligonucleotide within the Monovalent Streptavidin

The following example describes testing of the hybridization behavior of the oligonucleotide within the monovalent streptavidin from Example 1 and Example 2.

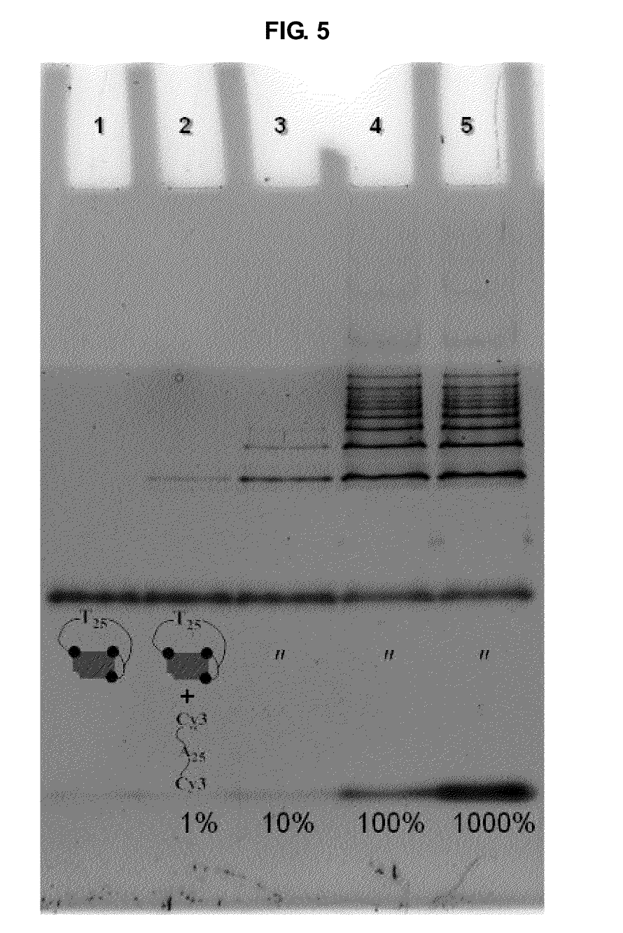

An initial intention was to test hybridization as an approach to incorporate a fluorescent dye for imaging applications. But when STV-(1-3) were mixed with their respective complementary oligonuclucleotides (dye-functionalized in the case of Cy3-A₂₅-Cy3 (SEQ ID NO: 19)), it was observed in PAGE experiments the appearance of an extensive ‘ladder’ (see e.g., FIG. 1B, lane 7), indicating an oligomerization process. It was hypothesized that double helix formation forced dissociation of one of the biotin moieties, triggering intermolecular crosslinking. Consistent with this mechanism, the extent of oligomerization observed is proportional to the amount of Cy3-A₂₅-Cy3 (SEQ ID NO: 19) added to STV-(1), i.e., sub-equivalent amounts of Cy3-A₂₅-Cy3 (SEQ ID NO: 19) resulted in a diminished ‘ladder’ because of excess STV-(1) binding to dissociated biotin moieties and thus ‘capping’ the propagation of the oligomerization process (see e.g., FIG. 5).

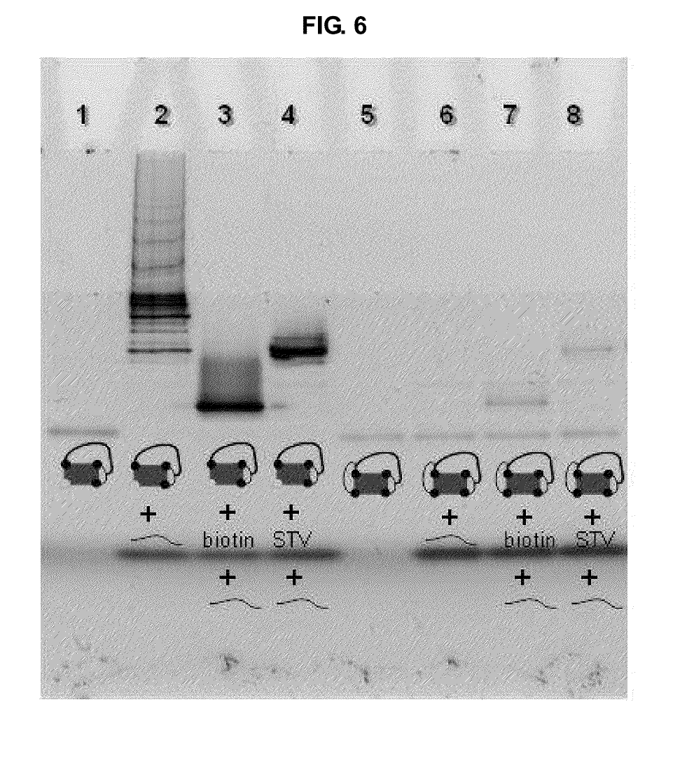

The oligomerization could be inhibited by introducing an additional biotin moiety at the mono-biotin end (3′ end) of the oligonucleotide, to give STV-(4) where the oligonucleotide is anchored with two biotins on both ends (see e.g., FIG. 6). This result is consistent with the hypothesis that the biotin moiety undergoing dissociation in STV-(1-3) is the single biotin moiety at the 3′-end. Oligomerization after hybridization can also be prevented either by ‘capping’ the dissociated biotin with an excess of free streptavidin or blocking incipient biotin binding sites by an excess of free biotin (see e.g., FIG. 6).

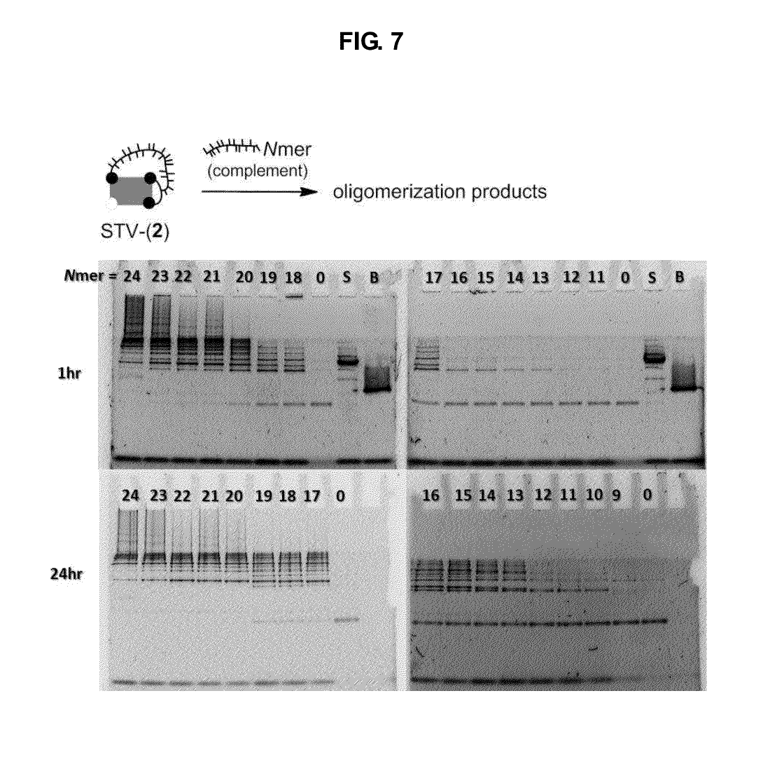

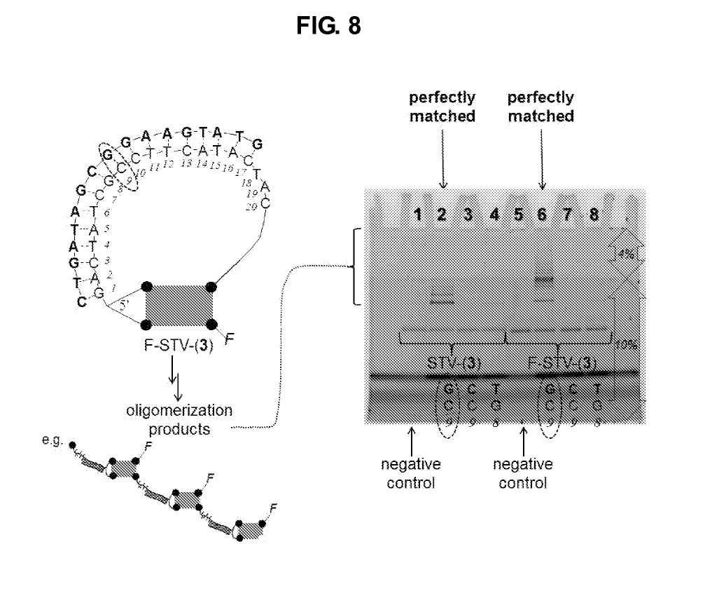

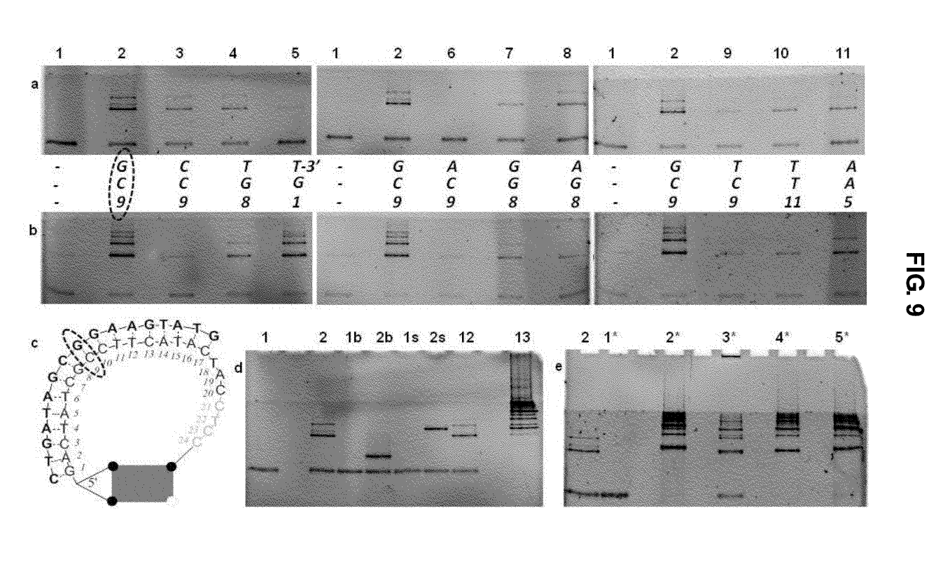

The biotin dissociation is likely driven by the relief of strain caused by the increased rigidity and shortening of the resulting double helix relative to the single strand (force acting on the biotin-streptavidin interaction can diminish the lifetime of the interaction). The oligomerization is also observed with shorter oligonucleotide complements (see e.g., FIG. 7), but with smaller oligomeric species produced over the time course of the experiment—which can be explained by a decrease in rate of the production of dissociated biotin, leading to preferential capture by starting material and a higher chance of cyclic oligomer formation (for example, in FIG. 7, c.f. lane 24 with lane 20 at 24 hrs where, importantly, all starting material has been consumed, but, lane 24 is more weighted towards larger oligomeric species whilst lane 20 is weighted towards shorter oligomeric species). Hindrance of large oligomer formation was also observed when blocking the single vacant biotin binding site of STV-(3) (see e.g., FIG. 8, cf. lane 2 with lane 6). This is due to the dissociated biotin moiety of F-STV-(3) having only the option of crosslinking to another biotin-dissociated species and not the option of being ‘capped’ by another F-STV-(3).

### Example 5

### Sensitivity of the Oligomerization to Single Base Mismatches

The sensitivity of the oligomerization to single base mismatches, a desirable attribute for any oligonucleotide detection system, was studied. Methods are according to Example 1, Example 2, and Example 4 unless otherwise indicated.

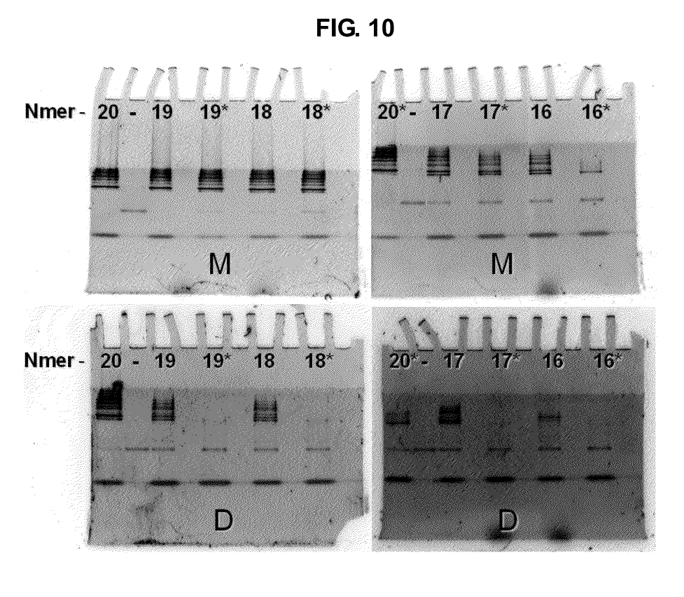

Fully complementary oligonucleotides (targets′) of different lengths and their single mismatch counterparts against STV-(2) (the ‘probe’) were screened and results were consistent with the following trade-off: long oligonucleotides caused opening more rapidly, while shorter oligonulcleotides were more sensitive to single-point mismatches. For example, for a 17-nucleotide ‘target’ strand the forced biotin dissociation process took three days for ˜100% dissociation (see e.g., FIG. 9e) whilst for the 24-nucleotide ‘target’ strand the process was relatively rapid at about 15 mins for ˜100% dissociation), but with the 17-mer ‘target’ more sensitive to the mismatch. From these results (see e.g., FIG. 10), 17-nucleotide long ‘targets’ were chosen to investigate the effect of various mismatches on the biotin dissociation and oligomerization products for the STV-(2), STV-(3), and F-STV-(3) ‘probes’.

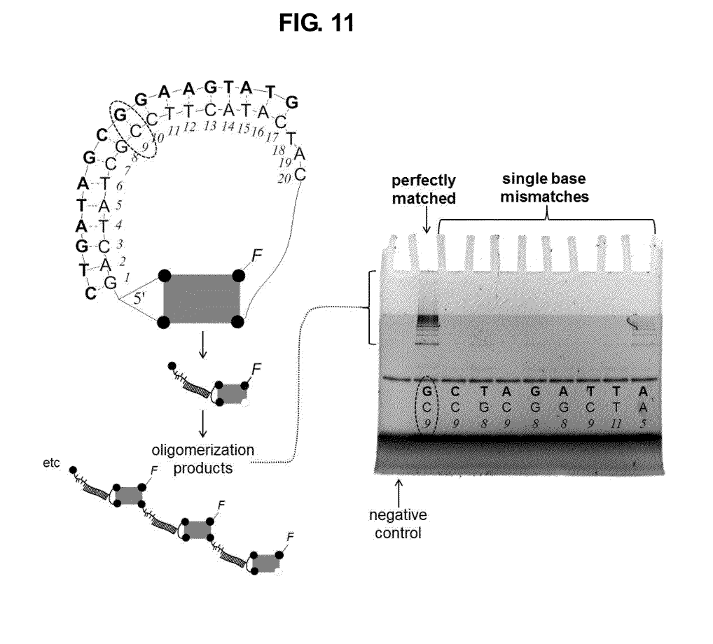

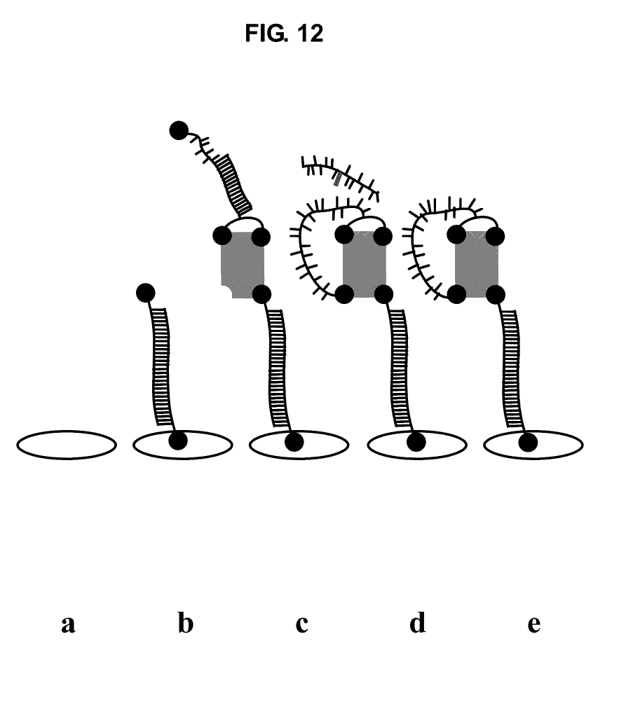

STV-(3) and F-STV-(3) showed high sensitivity for 15 min long, 37° C. incubation times for various single base mismatches in the 17 mer ‘target’ strand (see e.g., FIG. 8; FIG. 11). STV-(2), which contains a longer (by four bases) tris-biotinylated oligonucleotide, was less sensitive to single base differences than STV-(3) (compare FIG. 9a with FIG. 9b). Mismatch sensitivity was largely diminished (i.e., the rate of biotin dissociation of the perfectly matched compliment and that containing a single base difference was similar) if ‘target’ base-pairing started from the 3′-terminus of the ‘probe’ (note—FIG. 8 shows base-pairing starting from the 5′-terminus of the probe sequence) (cf. FIG. 10). Over longer time periods, observable mismatch sensitivity is gradually diminished due to all samples moving towards equilibrium, i.e., all starting material is oligomerized.

### Example 6

### Monobiotinylated-Oligonucleotide Handle

Incorporating an additional monobiotinylated-oligonucleotide provides a useful handle, for example, if there is a need to attach the reagent to a solid support, or as a spatial address in various microarray applications.

Methods are according to Example 1, Example 2, Example 4, and Example 5 unless otherwise indicated.

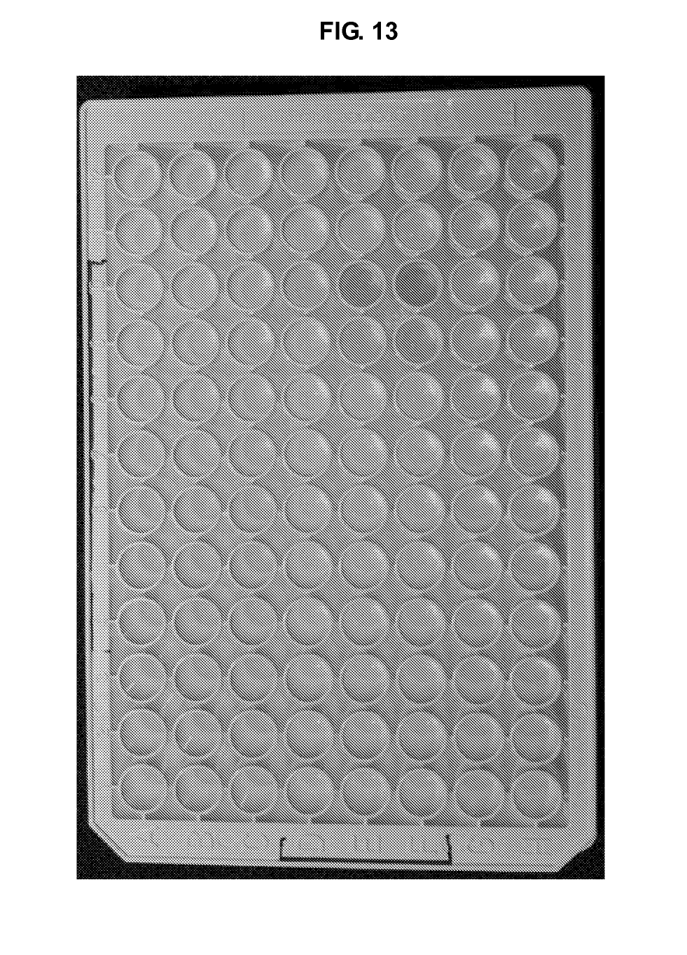

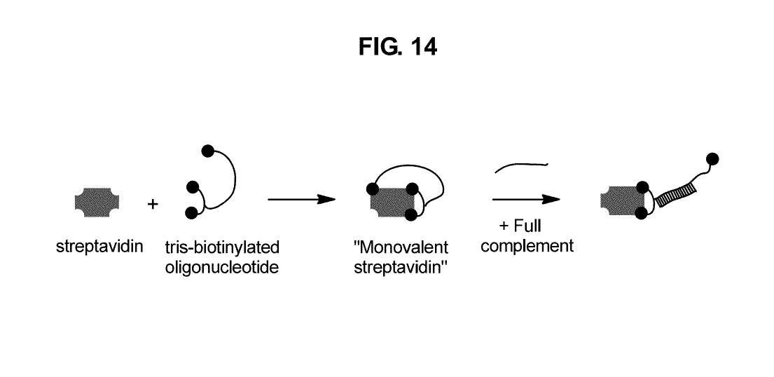

As a demonstration, the monovalent streptavidin STV-(3) was attached to streptavidin-coated plates, and a complementary oligonucleotide was detected specifically over a single-point mutation, within 15 minutes at room temperature, via labelling the dissociated biotin with HRP-streptavidin conjugate (see e.g., FIG. 12; FIG. 13). Perfectly matched oligonucleotides trigger dissociation of the biotin-streptavidin interaction at higher rates relative to SNPs (see e.g., FIG. 14).

Such process can be used for direct detection of short oligonucleotides of clinical significance.

### Example 7

### Bis-Biotinylated Oligonucleotide B2

The following example shows generation and testing of bis-biotinylated oligonucleotide. Methods are according to Example 3 unless otherwise specified.

For comparison, the bis-biotinylated oligonucleotide B2 was subjected to the same series of experiments as B3. When one equivalent of B2 was combined with STV, discrete STV-(B2)n species were formed (where n is 1 or 2). Notable was the absence of any detectable dimers or higher order oligomers. On heating the products, at 70° C. for 20 mins, a redistribution of the quantity of each species formed occurred. Before heating, the n=2 products are major and the n=1 are minor; and after heating, there is an equal distribution between n=1 and n=2.

In contrast, when ten equivalents of STV are combined with one equivalent of B2 (at room temperature) the product distribution is reversed, i.e., n=1 predominates with n=2 as the minor product. Combining STV with two equivalents of B2 produces solely n=2 species at room temperature and at 70° C. 260/280 ratios are used to verify stoichiometry of products. Similar to the STV-B3 complex, input of the ligand complementary strand results in a ring opening of the B2 ligand and the formation of cross-linked species (including dimers. In contrast, when duplex B2 is added directly to STV (1:1), only higher order oligomers are obtained (i.e., no dimers).

### Example 8

### Ring Opening of STV-B3

The following example shows ring opening of STV-B3. Methods are according to Example 3 and Example 7 unless specified otherwise.

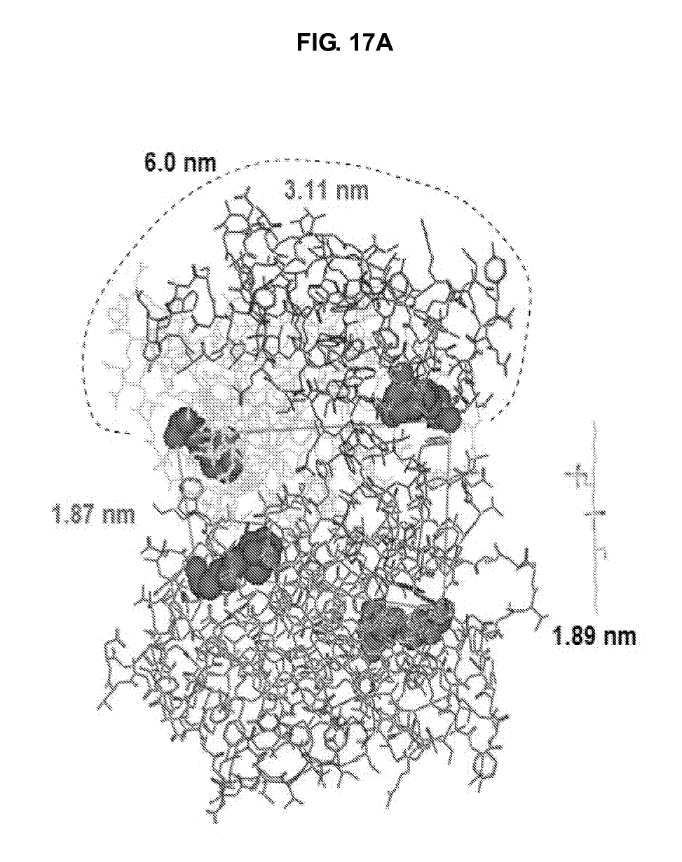

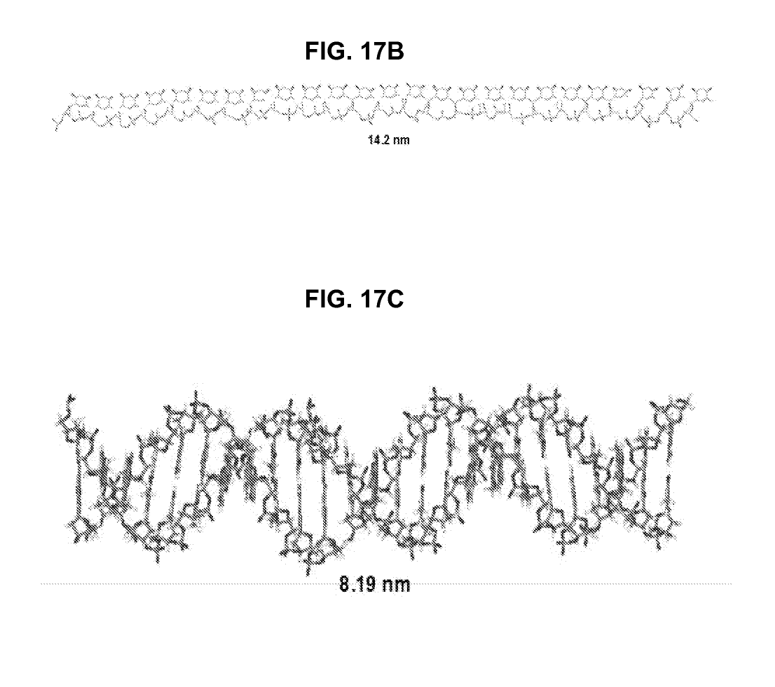

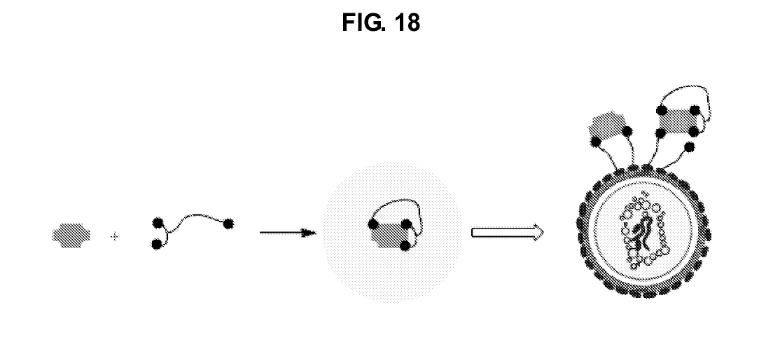

When STV-B3 was exposed to the full Watson-Crick base-pair oligonucleotide complement of the oligonucleotide sequence of B3, i.e. A25 (labeled with Cy5 at both the 5′ and 3′ ends), a ring opening occurred. The ring opening was the dissociation of a biotin moiety due to the strain placed on the complexed ligand by the more rigid structure of a duplex, relative to a single strand, coupled with the inherent shortening of the linker (see e.g., FIG. 17, compare b and c). Ring opening was evidenced by the disappearance of the STV-B3 species on the addition of (Cy3)A25(Cy3) (SEQ ID NO: 19) (50% gone after 5 mins, and 100% gone after 25 mins) and the appearance of higher order species, as observed by HPLC.

Therefore, the duplex formation results in the displacement of at least one of B3's three biotins, resulting in the STV oligomers corresponding charge-wise to (STV)n(B3)3 and (STV)m(B3)4 and higher order oligomers. When the preformed duplex of B3•(Cy3)A25(Cy3) is mixed in a 1:1 ratio with STV, no STV-B3 species is observed, only higher order species (above (STV)n(B3)3).

### Example 9

### STV-B3*

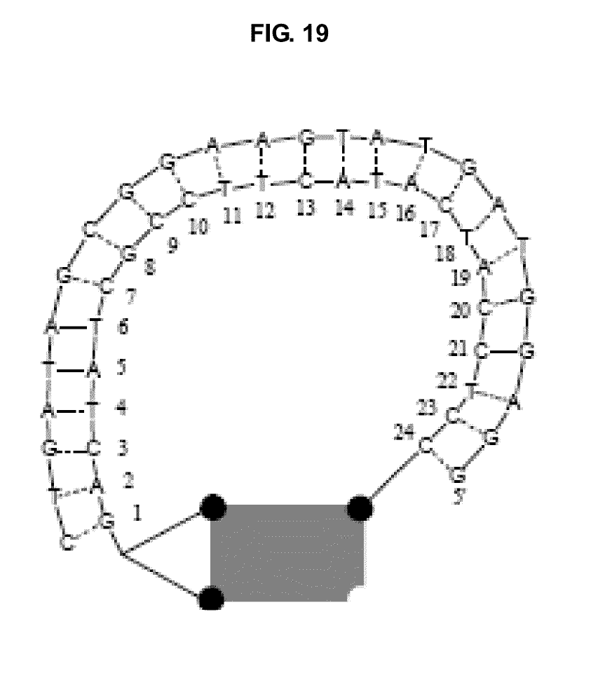

The following example describes generation and testing of a monovalent streptavidin STV-B3* closed structure (see e.g., FIG. 19) and ring opening. Methods are according to Example 3, Example 7, and Example 8 unless specified otherwise.

A tridentate biotin was synthesized using DNA. The use of tridentate biotin linker blocked three quarters of streptavidin sites by mixing equimolar amounts of linker and streptavidin.

The reaction of SW with 5′-dualbiotin-GAC TAT CGC CTT CAT ACT ACC TCC-monobiotin-3′ (SEQ ID NO: 14) (B3*) yielded STV-B3* (i.e., when 1 microM STV and 1 microM B3* were combined in equimolar amounts at 25° C., a one-to-one STV-B3* complex was obtained).

Results showed that, analogous to STV-B3 (see e.g., FIG. 19A and FIG. 19B), reaction of SW with 5′-dualbiotin-GAC TAT CGC CTT CAT ACT ACC TCC-monobiotin-3′ (SEQ ID NO: 14) (B3*) provided STV-B3*. Identification of species was made from IE-HPLC trace titrations and comparison to known standards, along with UV-vis data such as 260 nm/280 nm to qualify DNA/protein ratio. Thus is shown all biotins in the complex were bound to the same STV, which yielded a monovalent streptavidin species.

Oligonucleotides were commercially manufactured by Integrated DNA Technologies Inc. (Coralville, Iowa). The biotinylated oligonucleotides are as follows. B3*=5-/52-Bio/GAC TAT CGC CTT CAT ACT ACC TCC/3BioTEG/-3 (SEQ ID NO: 14). The 52-Bio is a 5′-end dual-biotin modification and 3BioTEG is a 3′-end monobiotin modification.

STV-B3* assembly: Lyophilized B3* was resuspended in distilled, deionized water (Mediatech, Cat. No. 46-000-CM, Lot No. 46000046) to give a stock solution concentration of 100 pM. Lyophilized streptavidin (ThermoFisher, Cat. No. 21135, Lot. No. LI150275) was resuspended in distilled, deionized water to give a stock solution concentration of 10 mg/mL. B3 stock solution (2.5 microL, 0.25 nmol) was added to buffer (250 microL of 20 mM TRIS, pH 7.2, 150 mM NaCl). Streptavidin stock solution (1.325 microL, 0.25 nmol) was added to buffer (250 microL of 20 mM TRIS, pH 7.2, 150 mM NaCl). The buffered B3 solution was rapidly added and mixed with the diluted streptavidin solution (250 microL). The solution was ready immediately for purification.

Results showed that when STV-B3* was exposed to the full Watson-Crick base-pair oligonucleotide complement of the oligonucleotide sequence of B3*, a ring opening occurred. Ring opening was the disassociation of a biotin moiety due to the strain placed on the complexed ligand by the more rigid structure of a duplex, relative to single strand, coupled with inherent shortening of the linker. The B3* ligand allows for investigation and optimization of factors, such as length and mismatches, effect biotin dissociation as triggered by the complementary oligonucleotide input (i.e., target strand).

### Example 10

### Additional Complexes

The following example describes generation and testing of various STV-B complexes. Methods are according to Example 3, Example 7, Example 8, and Example 9 unless specified otherwise.

(STV-(B3)2). According to STV-B3 synthesis, above, the addition of larger amounts of B3 (up to 10 equivalents) relative to STV results in the major product STV-(B3)2 (>50% yield).

STV-(B2)1 was made in analogous fashion to STV-B3* above, wherein, B2=5-/5BioTEG/TTT TTT TTT TTT TTT TTT TTT TTT T/3BioTEG/-3 (SEQ ID NO: 13) and 3BioTEG is a 3′-end monobiotin modification.

STV-(B2)2 was made in analogous fashion to STV-B3* above. B2=5-/5BioTEG/TTT TTT TTT TTT TTT TTT TTT TTT T/3BioTEG/-3 (SEQ ID NO: 13) and 3BioTEG is a 3′-end monobiotin modification.

STV-B4 assembly was made in an analogous fashion to STV-B3*, above. B4=5-/52-Bio/GAC TAT CGC CTT CAT ACT ACC TCC/iBiodT//3Bio/-3 (SEQ ID NO: 16), where 52-Bio is a 5′-end dual-biotin modification; 3Bio is 3′-end monobiotin modifications; and iBiodT is a biotin functionalized thymine nucleotide.

Higher order oligomers, such as (STV)m(B3)3 and (STV)n(B3)4 were generated.

### Example 11

The following example describes a “switch” that converts monovalent streptavidin to divalent streptavidin. Methods are according to Example 3, Example 7, Example 8, Example 9, and Example 10 unless specified otherwise.

As shown above, providing a complementary oligonucleotide to a single strand of the oligonucleotide linker pulls off one biotin, freeing a binding site on the streptavidin, where the free biotin end binds with a streptavidin with a label.

A nucleic acid, complementary to a biotin linker, removed one biotin by straightening the tether (see e.g., FIG. 15). Other linked structures on FIG. 15 also open up. Linker possibilities include locked nucleic acid (LNA) also known as locked sugars, inaccessible RNA, which is a modified RNA nucleotide. Another possibility for a linker is peptide nucleic acid (PNA). PNA is similar to DNA or RNA, but is not known to occur naturally, and unlike DNA or RNA, has an uncharged backbone. Also, the DNA for the longest arm can also be an organic linker. Methods for preparation of the monovalent streptavidin are according to Example 1 unless specified otherwise.

If STV-B3 is exposed to the full Watson-Crick base-pair oligonucleotide complement to the oligonucleotide sequence of B3, a ring opening occurs due to the strain placed on the complexed ligand by the more rigid duplex structure. This is shown by the disappearance of the STVB3 species on the addition of (Cy3)A25(Cy3) (SEQ ID NO: 19) (50% gone after 5 mins, and 100% gone after 25 mins) and the appearance of higher order species, as observed by HPLC. Therefore, the duplex formation results in the displacement of one of B3's three biotins, resulting in the STV oligomers corresponding charge-wise to (STV)n(B3)3 and (STV)m(B3)4 and higher order oligomers with the B3 ligand in a STV-STV bridging binding mode. When the preformed duplex of B3•(Cy3)A25(Cy3) (SEQ ID NO: 19) is mixed in a 1:1 ratio with STV, no STV-B3 species is observed, only higher order species (above (STV)n(B3)3).

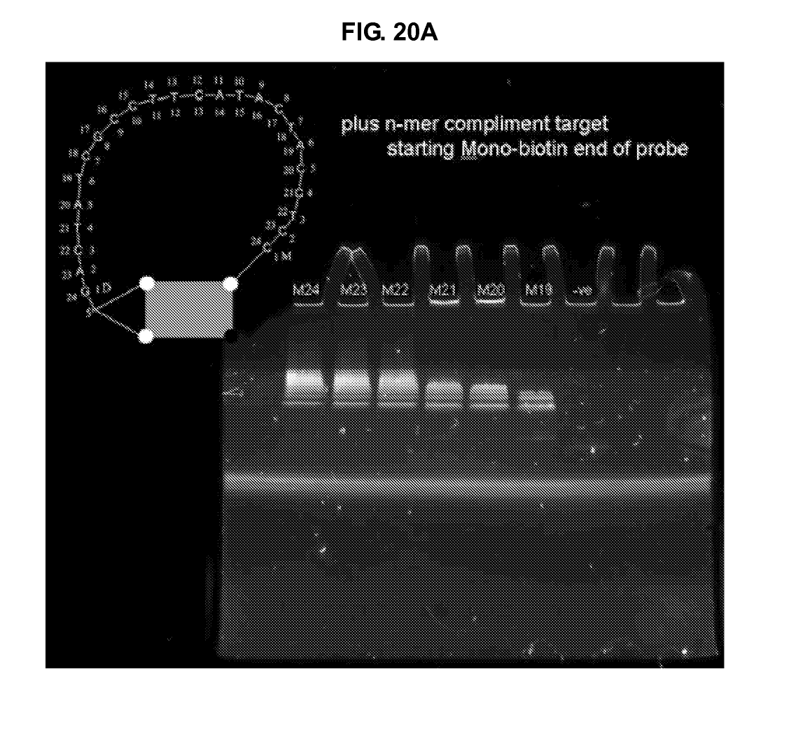

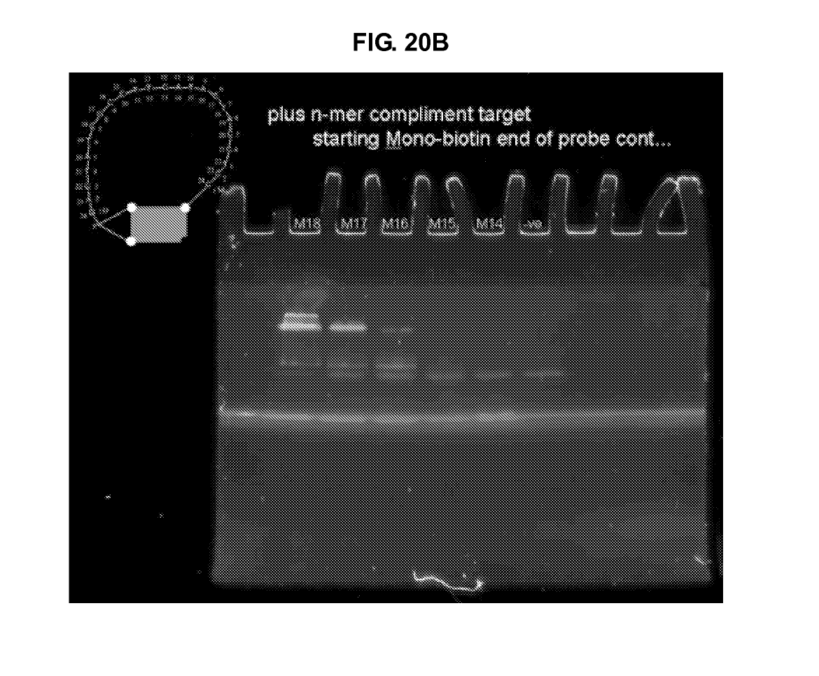

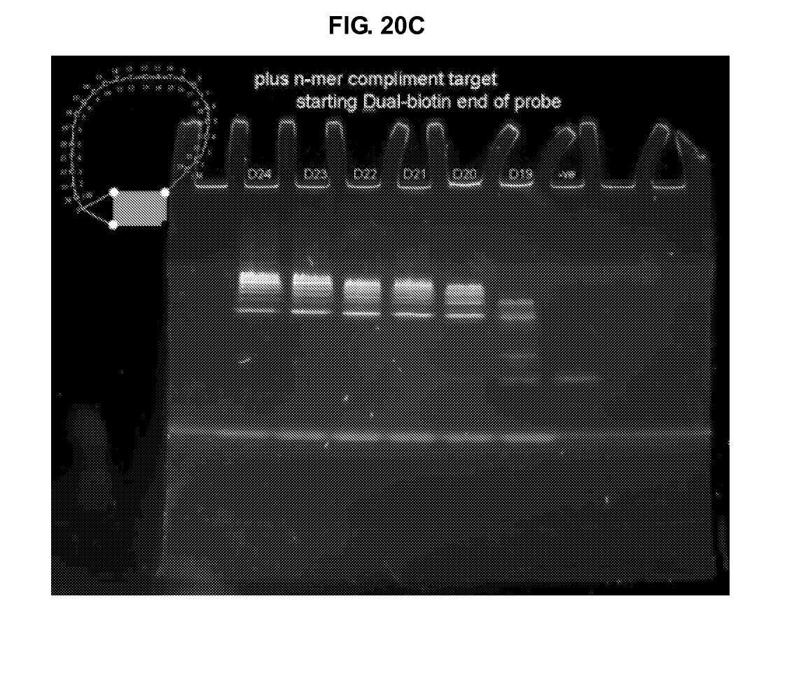

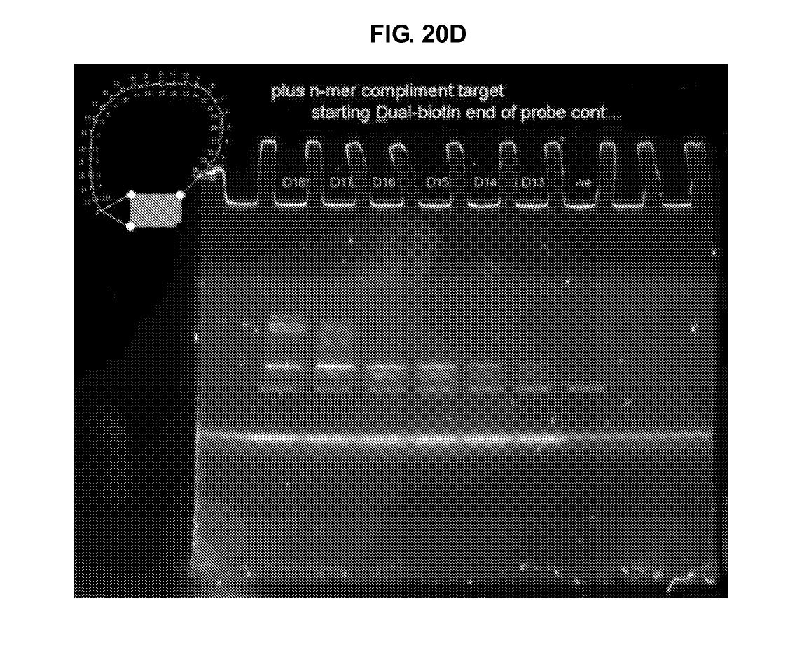

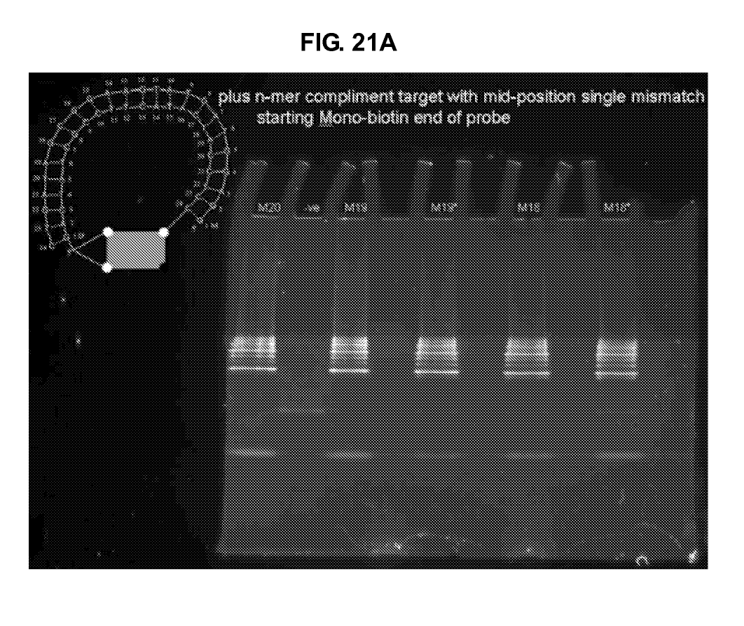

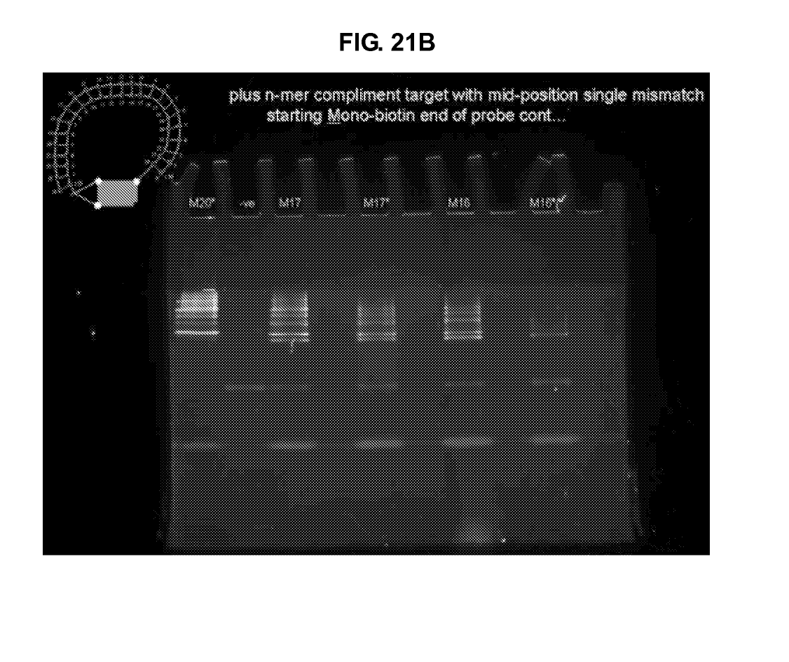

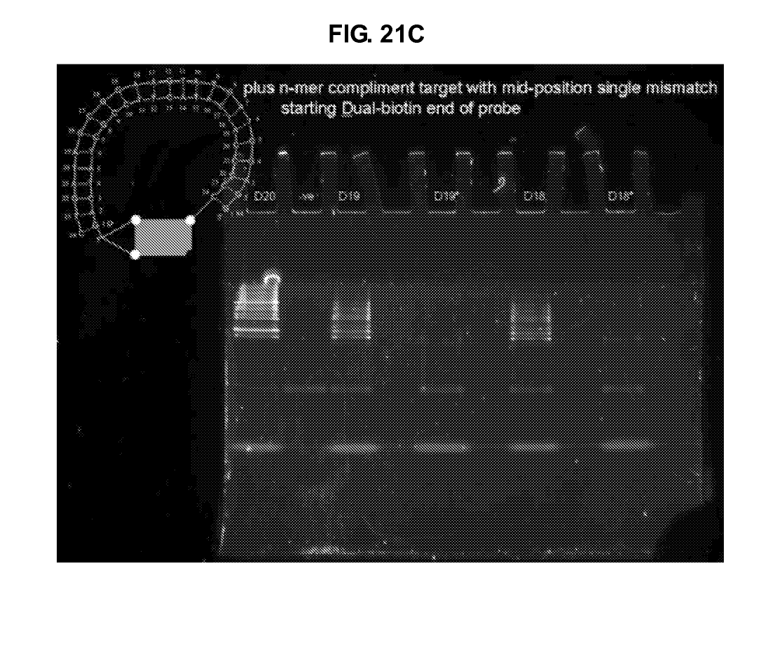

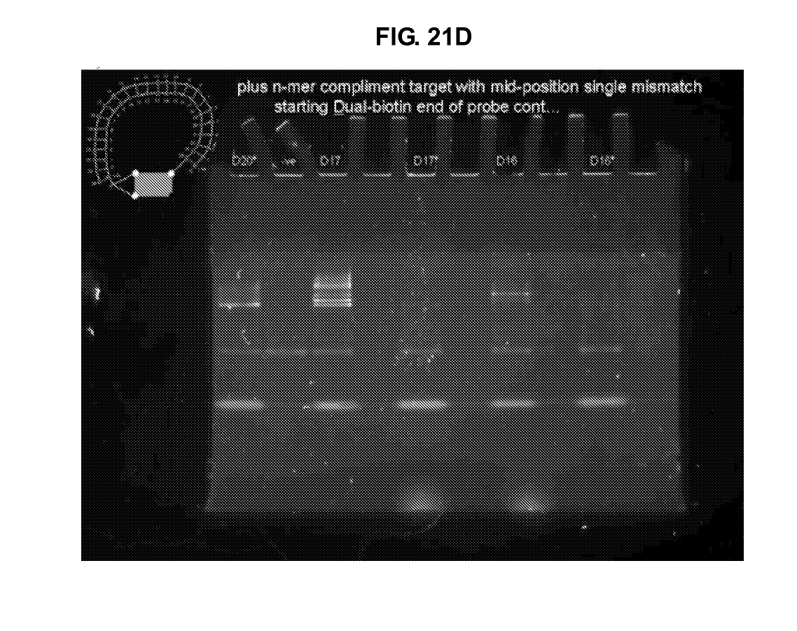

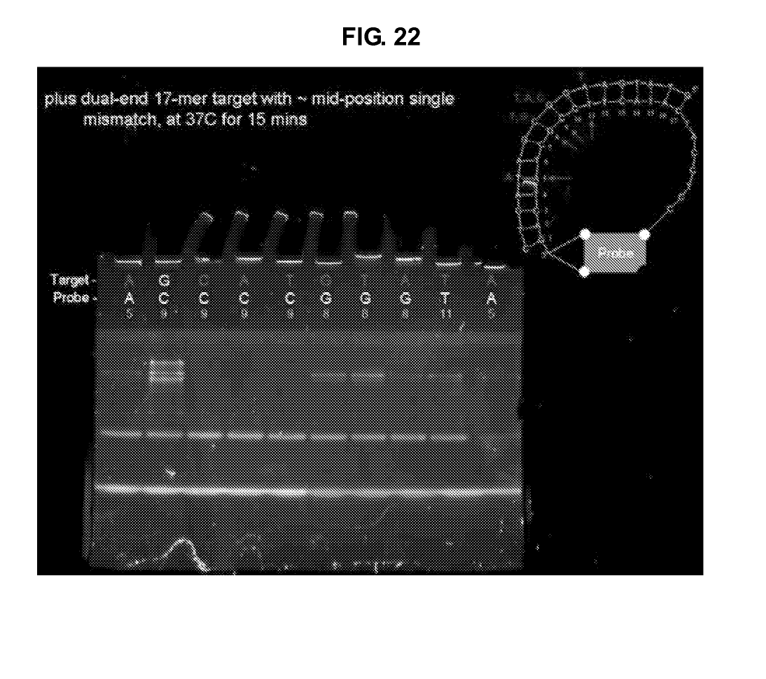

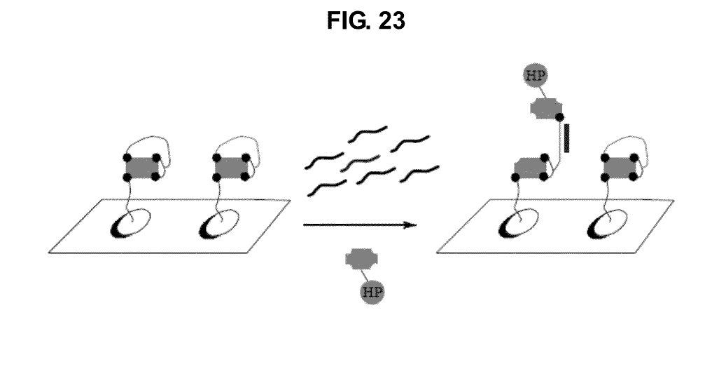

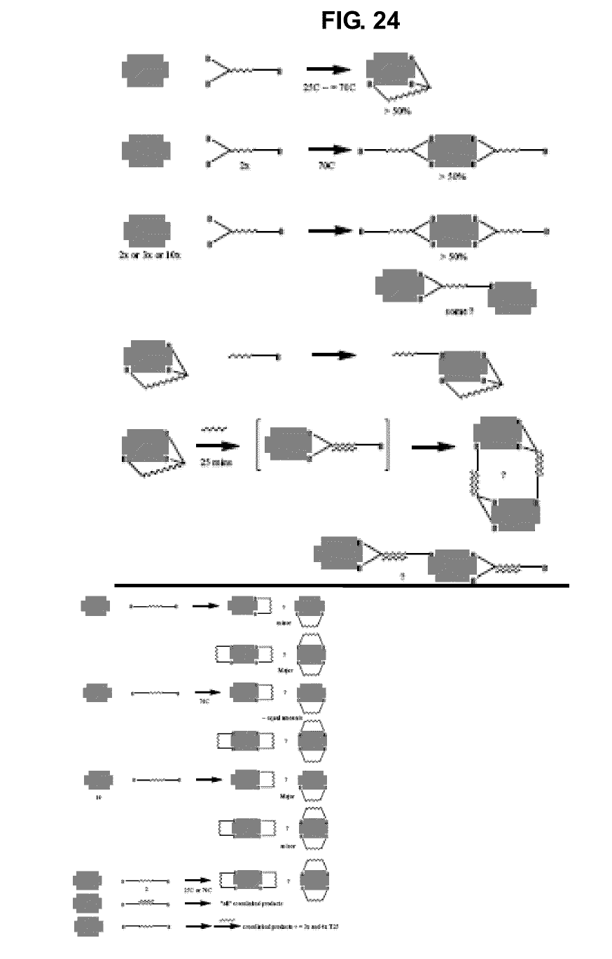

More specifically, see FIG. 19 for an example of a “ring-closed STV-B compound. FIG. 20, FIG. 21, FIG. 22, FIG. 23 and FIG. 24 show several examples that demonstrate complimentary strand-induced ring opening.

The effect of input (target) oligonucleotide length and hybridization position (i.e. from the dual biotin end (D) or from the mono biotin end (M)) were investigated.

All inputs from M15 mer to M24 mer (numbered from the Mono-biotin end) produced a change relative to the zero input sample (i.e. no input added—negative control), as analyzed by PAGE.

Below M15 mer no detectable differences were observed. The extent of higher order species increased dramatically as the length of the MNmer increased, as seen in FIG. 20A and FIG. 20B.

The inputs (or target) strands from D13 mer to D24 mer (numbered from the Dual-biotin end, see FIG. 19 for Dual-end numbering scheme) produced a change relative to the zero input sample, as observed by PAGE.

However, significant higher order species seen in D17 mer and above, see FIG. 20C and FIG. 20D. This decrease in the degree of polymerization can be explained in terms of kinetics. The shorter target oligonucleotides do not bind as strongly to the complexed B3^{*} ligand and hence the net dissociation of biotin moieties is slower. This results in the effective concentration of exposed biotins at any one time to be lower, therefore increasing the probability of, for example, cyclic dimer, cyclic trimer, etc, formation.

### Example 12

The sensitivity of the biotin dissociation process to single mismatches was analyzed. Methods are according to Example 3, Example 7, Example 8, Example 9, Example 10, and Example 11 unless specified otherwise.

Guided by results shown in FIG. 20 above, a 16-to-20 mers was selected for initial single mismatch studies. The mismatches were selected to occur at a position in the middle of the target oligonucleotide. For MNmer target strands, an AA mismatch at position 11 was used for N=20; TT at position 10 for N=19, and AA at position 9 for N=16, 17, and 18. For DNmer target strands, a CC mismatch at position 10 for N=19 and 20, and a CC mismatch at position 9 N=16, 17 and 18, was incorporated.

Results for MNmers showed significant sensitivity to the single mismatch only for the M16 mer. Above M16 mers, sensitivity quickly diminished (see e.g., FIG. 21A-B). In contrast, results for DNmers showed great sensitivity for all lengths (D16 merto-D20 mer) (see e.g., FIG. 21C-D).

From the results shown in FIG. 21 above, the target strand D17 mer was selected for further single mismatch sensitivity studies. In the next set of experiments, sensitivity to specific mismatches were screened for all possible mismatch combinations involving G, C, A, and T. PAGE results are shown in FIG. 22. The full-complement target versus target strands carrying a single mismatch were easily discerned by PAGE. “Perfect” discrimination was achieved with all mismatches involving cytosine. Slight oligomerization was observed for other mismatches. Promisingly, the GT mismatch, the most likely to be an insensitive mismatch, was able to be easily discerned (compare lanes 2 and 7 in FIG. 22).

For comparison, the bis-biotinylated oligonucleotide B2 was subjected to the same series of experiments as B3. When one equivalent of B2 was combined with STV, discrete STV-(B2)n species were formed (where n is 1 or 2). Notable was the absence of any detectable dimers or higher order oligomers. On heating the products, at 70 C for 20 mins, a redistribution, of the quantity of each species formed, occurs—before heating the n=2 products are major and the n=1 are minor, and after heating there is an equal distribution between n=1 and n=2. In contrast, when ten equivalents of STV are combined with one equivalent of B2 (at room temperature) the product distribution is reversed, that is n=1 predominates with n=2 as the minor product. Combining STV with two equivalents of B2 produces solely n=2 species at room temperature and at 70 C. Importantly, and similar to the STV-B3 complex, input of the ligand complementary strand results in a ring opening of the B2 ligand and the formation of cross-linked species (including dimers). In contrast, when duplex B2 is added directly to STV (1:1) only higher order oligomers are obtained (i.e. no dimers).

### Example 13

### Quantification of Biotinylation

The following example shows use of a monovalent Streptavidin reagent to quantify the extent of biotinylation of a target.

In one experiment, bovine serum albumin (BSA) protein was biotinylated. Subsequently, biotinylated BSA was reacted with a monovalent Streptavidin reagent and a sample of less than 10 pM was quantified with standard polyacrylamide gel electophoresis (PAGE) (8%) with SYBR Gold stain (see e.g., FIG. 25A). Exemplary results are shown in FIG. 25B.

In another experiment, monovalent Streptavidin reagent was used to quantify biotinylation of the therapeutic antibody Rituxan. Disulfide reduced rituximab was reacted with 50 equivalents of maleimide-PEG11-biotin (See e.g., FIG. 26A). 10 picomole samples of biotinylated-antibody were reacted with 1 to 10 equivalents of monovalent streptavidin (see e.g., FIG. 26A) and analyzed according to the Native PAGE (5%) with SYPRO® Ruby protein gel stain (10 pM of biotinylated-antibody used per lane) (see e.g., FIG. 26B). Densitometry analysis Native Page results showed the distribution of biotinylated product (see e.g., FIG. 26C).

What is claimed is:

1. A composition comprising:
a streptavidin molecule comprising four biotin binding sites;
at least two biotin molecules; and
at least a first linker;
wherein,
the first linker connects the first biotin and the second biotin,
the first biotin is bound to a first biotin binding site of the streptavidin, and
the second biotin is bound to the second biotin binding site of the streptavidin; and
optionally, the composition further comprises a third biotin and a second linker, wherein the second linker connects the third biotin to one or more of the first biotin, the second biotin, or the first linker, and the third biotin is bound to a third biotin binding site of the streptavidin.

2. The composition of claim 1, wherein none of the linked biotins bind to a second streptavidin.

3. The composition claim 1, wherein the linker comprises a nucleic acid, an organic compound, or a combination thereof.

4. The composition of claim 3, wherein the linker comprises a DNA, an RNA, a locked nucleic acid (LNA), an inaccessible RNA, a peptide nucleic acid (PNA), or a combination thereof.

5. The composition of claim 1, wherein
(i) the first linker is at least about 1.8 nm, at least about 1.9 nm, at least about 2.0 nm, at least about 2.5 nm, at least about 2.6 nm, at least about 2.7 nm, at least about 2.8 nm, or at least about 2.9 nm in length; or
(ii) the second linker is at least about 6 nm, at least about 7 nm, at least about 8 nm, at least about 9 nm, at least about 10 nm, at least about 11 nm, at least about 12 nm, at least about 13 nm, at least about 14 nm, at least about 15 nm, at least about 16 nm, at least about 17 nm, at least about 18 nm, at least about 19 nm, or at least about 20 nm in length.

6. The composition of claim 1, wherein the streptavidin comprises an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12, or at least about 95% identical thereto and retaining or substantially retaining a high affinity for biotin.

7. A method of detecting a target molecule, the method comprising:
contacting a composition of claim 1 and a biotinylated target molecule under conditions where a biotin binding site of the composition can bind to the biotin of the biotinylated target molecule; and
detecting the composition bound to the biotinylated target molecule.

8. The method of claim 7, wherein the target molecule is attached to the surface of a cell.

9. The method of claim 7, wherein the composition comprises a detectable tag.

10. The method of claim 7, wherein the target molecule comprises an amino acid or a nucleic acid.

11. A method of detecting a nucleic acid in a sample comprising:
providing a composition of claim 1, wherein the composition comprises a streptavidin, at least three biotin molecules, a first linker, and a second linker comprising a nucleic acid complementary or substantially complementary to at least a portion of a target nucleic acid compound;
combining the composition and a sample that may contain the target nucleic acid compound under conditions that, if the target nucleic acid compound is present, the target nucleic acid binds to the complementary nucleic acid linker resulting in at least one biotin being dislodged from the streptavidin thereby exposing a streptavidin-biotin binding site;
contacting a labeled streptavidin with the composition; and
detecting presence or absence of the label;
wherein presence of the label indicates presence of the target nucleic acid compound in the sample.

12. The method of claim 11, wherein the target nucleic acid is a microRNA.

13. The method of claim 12, wherein the microRNA is about 20 to about 25 nucleotides in length.

14. The method of claim 12, wherein the microRNA is about 20, about 21, about 22, about 23, about 24, or about 25 nucleotides in length.

15. The method claim 12, wherein the nucleic acid of the second linker is the same or substantially the same length as the microRNA.

16. The method of claim 11, wherein:
if the target nucleic acid fully matches the nucleic acid of the second linker, the biotin binding site is exposed; and
if one or more mismatches exist between the target nucleic acid and the nucleic acid of the second linker, the biotin binding site is not exposed.

17. A method of determining biotinylation of a target molecule, the method comprising:
providing a sample comprising a biotinylated target molecule;
combining the sample and a composition according to claim 1 comprising monovalent streptavidin under conditions where the one available biotin binding site of the streptavidin in the composition can bind to a biotin of the biotinylated target molecule in the sample;
separating biotinylated target molecules according to differing numbers of monovalent streptavidin bound thereto; and
determining biotinylation level of the target molecule.

18. The method according to claim 17, wherein the biotinylated target molecule and the monovalent streptavidin are combined in a ratio of at least about 1:1, about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, or about 1:20.

19. The method according to claim 17, wherein:
a plurality of samples comprising a biotinylated target molecule is provided;
at least a portion of the plurality of samples are combined with different amounts of monovalent streptavidin;
biotinylated target molecules are separated according to differing numbers of monovalent streptavidin bound thereto.

20. The method of claim 17, wherein separating comprises polyacrylamide gel electrophoresis (PAGE).


SEQUENCE LISTING (19 sequences)

SEQ ID NO: 1 - protein, 159 aa, Artificial Sequence
  synthetic construct
DPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 2 - protein, 121 aa, Burkholderia pseudomallei
MLRRVKAGTGAPIDFSGTWKNELGSTMRIEQSGDSVSGTYESAVSENGGATSGQLSGYVDGDLIAFVVHWDQFQAITAWVGQGGPGASSDRINTLWQMTQQVEAGEEWASINAGADIFVKT

SEQ ID NO: 3 - protein, 124 aa, Burkholderia mallei
MEHVLRRVKAGTGAPIDFSGTWKNELGSTMRIEQSGDSVSGTYESAVSENGGATSGQLSGYVDGDLIAFVVHWDQFQAITAWVGQGGPGASSDRINTLWQMTQQVEAGEEWASINAGADIFVKT

SEQ ID NO: 4 - protein, 121 aa, Burkholderia pseudomallei
MLRRVKAGTGAPIDFSGTWKNELGSTMRIEQSGDSVSGTYESAVSENGGATSGQLSGYVDGDLIAFVVHWDQFQAITAWVGQGGPGASSDRINTLWQMTQQGEAGEEWASINAGADIFVKT

SEQ ID NO: 5 - protein, 127 aa, Burkholderia mallei
MQRLEHVLRRVKAGTGAPIDFSGTWKNELGSTMRIEQSGDSVSGTYESAVSENGGATSGQLSGYVDGDLIAFVVHWDQFQAITAWVGQGGPGASSDRINTLWQMTQQVEAGEEWASINAGADIFVKT

SEQ ID NO: 6 - protein, 155 aa, Burkholderia sp. E264
MKNAFVSLAIPGLLASSLLADSAYAANCQNPLGTWLNELGSTMTISTYSSSSGAISGAYKSPSGTSGQTFPLSGWFYAPSSASPGLDQVTLFTFSVNWNTPQAQYNSITTWSGTCRTTNGSPTIQALWYYTNAFGQYSWKHTNAGQDVFQPTGPQ

SEQ ID NO: 7 - protein, 127 aa, Burkholderia sp. E264
MQRLEHALRRVKVGTGTPIDFSGTWKNELGSTMRIEQSGDSVTGTYTSAVSEKGGSTSGDLIGYVDGNLIAFVVHWDQFQAITAWVGQCEPGTSDDRISTLWQMTHQVEAGDEWASINAGADTFVRA

SEQ ID NO: 8 - protein, 166 aa, Burkholderia mallei
MRCTIVLGIRAASPIKEALARPAPRPGRLPSIHRSGRRNMQRLEHVLRRVKAGTGAPIDFSGTWKNELGSTMRIEQSGDSVSGTYESAVSENGGATSGQLSGYVDGDLIAFVVHWDQFQAITAWVGQGGPGASSDRINTLWQMTQQVEAGEEWASINAGADIFVKT

SEQ ID NO: 9 - protein, 132 aa, Mycobacterium thermoresistibile
MVLQHISGLRKSAQSSGRPVDFNGTWLNELGSELHLEVDGQGRVTGTFQTAVGAPQPTQKFEVTGFVAGDVLAFCVNFGAYASLSSWVGQHTVEDGNEVIKAMWLLGRDIKDADEPTDLWSAVLTGASNFRR

SEQ ID NO: 10 - protein, 132 aa, Mycobacterium thermoresistibile; identical to SEQ ID NO: 9

SEQ ID NO: 11 - protein, 159 aa, Streptomyces lavendulae
DPSKESKAQAAVAEAGITGTWYNQLGSTFIVTANPDGSLTGTYESAVGNAESRYVLTGRYDSTPATDGSGTALGWTVAWKNNYRNAHSATTWSGQYVGGAEARINTQWLLTSGTTAANAWKSTLVGHDTFTKVKPSAASIDAAKKAGVNNGNPLDAVQQ

SEQ ID NO: 12 - protein, 160 aa, Streptomyces avidinii
MDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 13 - protein, 25 aa, Artificial Sequence
  Synthetic construct; 5′ dual-biotin modification of 52-Bio and a 3′ monobiotin modification of 3BioTEG on the synthetic sequence
TTTTTTTTTTTTTTTTTTTTTTTTT

SEQ ID NO: 14 - protein, 24 aa, Artificial Sequence
  Synthetic construct; 5′ dual-biotin modification of 52-Bio is a and a 3′ monobiotin modification of 3BioTEG on the synthetic sequence
GACTATCGCCTTCATACTACCTCC

SEQ ID NO: 15 - protein, 20 aa, Artificial Sequence
  Synthetic construct; 5′ dual-biotin modification of 52-Bio and a 3′ monobiotin modification of 3BioTEG on the synthetic sequence
GACTATCGCCTTCATACTAC

SEQ ID NO: 16 - protein, 24 aa, Artificial Sequence
  Synthetic construct; 5′ dual-biotin modification of 52-Bio and a biotin functionalized thymine nucleotide iBiodT, and 3′ monobiotin modification of 3Bio
GACTATCGCCTTCATACTACCTCC

SEQ ID NO: 17 - protein, 30 aa, Artificial Sequence
  Synthetic construct; 5′ monobiotin modification of 5BioTEG
CGGTTTTTTGTTCTTTGTTTTGTTCTTTGC

SEQ ID NO: 18 - protein, 30 aa, Artificial Sequence
  Synthetic construct; 5′ monobiotin modification of 5BioTEG
GCAAAGAACAAAACAAAGAACAAAAAACCG

SEQ ID NO: 19 - protein, 25 aa, Artificial Sequence
  Synthetic construct; 5′ and 3′ modification of the dye Cy3
AAAAAAAAAAAAAAAAAAAAAAAAA